(19) United States
(12) Patent Application Publication
(10) Pub. No.: US 2014/0219951 A1
(43) Pub. Date: Aug. 7, 2014

(54) AMIDO-AMINE DENDRIMER COMPOSITIONS

(71) Applicant: GENZYME CORPORATION, CAMBRIDGE, MA (US)

(72) Inventors: PRADEEP DHAL, Westford, MA (US); S. Randall Holmes-Farley, Arlington, MA (US); Chad Huval, Grand Coteau, LA (US); Steven C. Polomoscanik, Methuen, MA (US)

(73) Assignee: GENZYME CORPORATION, CAMBRIDGE, MA (US)

(21) Appl. No.: 14/248,523

(22) Filed: Apr. 9, 2014

Related U.S. Application Data
(63) Continuation of application No. 12/451,060, filed on Mar. 4, 2010, now abandoned.

(51) Int. Cl. A61K 31/785

(52) U.S. Cl. CPC A61K 31/785
USPC 424/78.37

(57) ABSTRACT

Amide compounds, amide polymers and compositions including amide compounds and amide polymers may be used to bind target ions, such as phosphorous-containing compounds in the gastrointestinal tract of animals. In some cases, the amide polymers may be amido-amine dendrimers that may be formed via a series of iterative reactions.

### FIELD OF THE INVENTION

This invention relates to amido-amine polymers for binding target ions, and more specifically relates to pharmaceutically acceptable compositions, amido-amine dendrimers, and amido-amine polymers or residues thereof for binding target ions.

### BACKGROUND OF THE INVENTION

Hyperphosphatemia frequently accompanies diseases associated with inadequate renal function such as end stage renal disease (ESRD), hyperparathyroidism, and certain other medical conditions. The condition, especially if present over extended periods of time, leads to severe abnormalities in calcium and phosphorus metabolism and can be manifested by aberrant calcification in joints, lungs, and eyes.

Therapeutic efforts to reduce serum phosphate include dialysis, reduction in dietary phosphate, and oral administration of insoluble phosphate binders to reduce gastrointestinal absorption. Many such treatments have a variety of unwanted side effects and/or have less than optimal phosphate binding properties, including potency and efficacy. Accordingly, there is a need for compositions and treatments with good phosphate-binding properties and good side effect profiles.

### BRIEF SUMMARY OF THE INVENTION

In one aspect, the present invention relates to amido-amine compounds, amido-amine polymers and/or pharmaceutical compositions comprising, at least in part, amido-amine compounds (including amido-amine dendrimers) or residues thereof derived from a multi-amine monomer and a multifunctional monomer comprising two or more amine-reactive groups. The amido-amine compounds can be crosslinked to form amido-amine polymers. Compositions can comprise one or more amido-amine compounds and/or amido-amine polymers or residues thereof. Several embodiments of the invention, including this aspect of the invention, are described in further detail as follows. Generally, each of these embodiments can be used in various and specific combinations, and with other aspects and embodiments unless otherwise stated herein.

In addition to the amido-amine compounds and amido-amine polymers of the present invention as described herein, other forms of the amido-amine polymers and amido-amine compounds are within the scope of the invention including pharmaceutically acceptable salts, solvates, hydrates, prodrugs, polymorphs, clathrates, and isotopic variants and mixtures thereof of the amido-amine compounds and/or amido-amine polymers.

In addition, amido-amine compounds and amido-amine polymers of the invention may have optical centers, chiral centers or double bonds and the amido-amine compounds and amido-amine polymers of the present invention include all of the isomeric forms of these compounds and polymers, including optically pure forms, racemates, diastereomers, enantiomers, tautomers and/or mixtures thereof

The invention provides methods of treating an animal, including a human. The method generally involves administering an effective amount of one or more amido-amine compounds or amido-amine polymers or a composition (e.g., a pharmaceutical composition) comprising the same to the animal as described herein.

In some embodiments, the invention is, consists essentially of, or comprises an amido-amine compound or residue thereof, an amido amine polymer or a pharmaceutical composition comprising an amido-amine compound or residue thereof or an amido-amine polymer. In some embodiments, the amido-amine compound is derived from two or more compounds or comprises a residue of two or more compounds where the compounds comprise a multi-amine and a multifunctional compound, where the multifunctional compound comprises two or more amine-reactive groups. In some embodiments, the amido-amine compound comprises an amido-amine dendrimer and, in some embodiments, may be formed via a series of alternating reactions.

In some embodiments, the invention is, consists essentially of, or comprises an amido-amine compound or residue thereof or an amido-amine polymer that comprises at least one amido-amine compound or residue thereof, where the amido-amine compound is derived from compounds according to the following Formulas I and II:

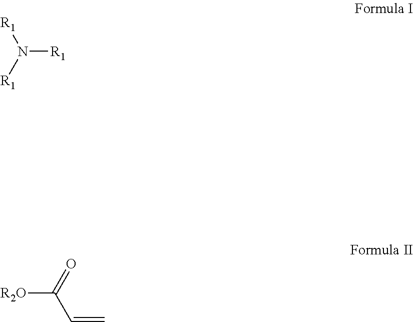

where R₁ independently represents a hydrogen radical, —RNH₂, —R—N—(R—NH₂)₂ or —R—N—(R—N—(R—NH₂)₂)₂, wherein R independently represents a branched or unbranched, substituted or un-substituted alkyl radical, with the proviso that at least one R₁ is not a hydrogen radical and R₂ independently represents a hydrogen radical or a branched or unbranched, substituted or un-substituted alkyl radical.

In some embodiments, the invention is, consists essentially of, or comprises an amido-amine compound or residue thereof or an amido-amine polymer that comprises at least one amido-amine compound or residue thereof, where the amido-amine compound is represented by the following Formula III:

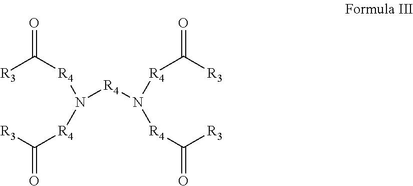

where R₃ independently represents a group represented by the following Formula IV:

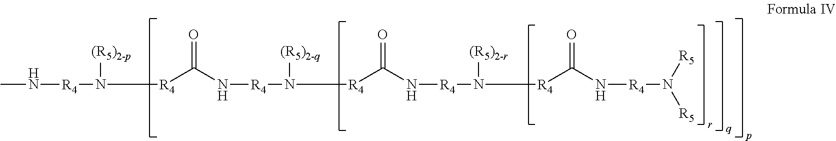

where p, q and r independently represent an integer from 0-2; R₄ independently represents

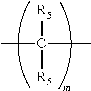

where m independently represents an integer from 1-20; R₅ independently represents a hydrogen radical; a substituted or un-substituted alkyl radical; a substituted or un-substituted aryl radical; or R₅ and a neighboring R₅ together represent a link or links comprising a residue of a crosslinking agent, a substituted or un-substituted alicyclic radical, a substituted or un-substituted aromatic radical, or a substituted or un-substituted heterocyclic radical; or R₅ represents a link with another compound or a residue thereof.

In some embodiments, the invention is, consists essentially of, or comprises an amido-amine compound or residue thereof or an amido-amine polymer that comprises at least one amido-amine compound or residue thereof, where the amido-amine compound is represented by the following Formula VIII:

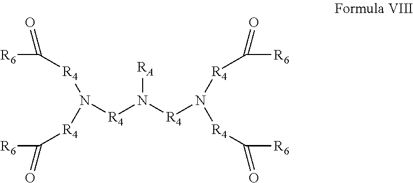

R₅ independently represents a group represented by the following Formula IX:

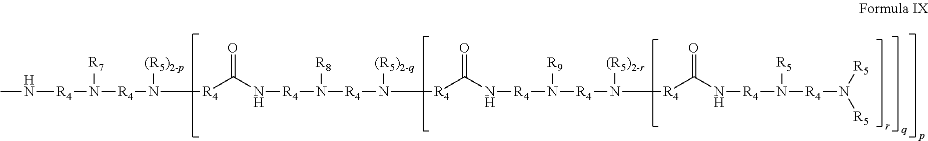

where p, q and r independently represent an integer from 0-2; R₄ independently represents:

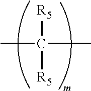

where m independently represents an integer from 1-20; R₅ independently represents a hydrogen radical; a substituted or un-substituted alkyl radical; a substituted or un-substituted aryl radical; or R₅ and a neighboring R₅ together represent a link or links comprising a residue of a crosslinking agent, a substituted or un-substituted alicyclic radical, a substituted or un-substituted aromatic radical, or a substituted or un-substituted heterocyclic radical; or R₅ represents a link with another compound or a residue thereof; R_{A} independently represents an R₅ group or a —R₄—CO—R₆ group; R₇ independently represents an R₅ group or independently represents a group according to the following Formula XIV:

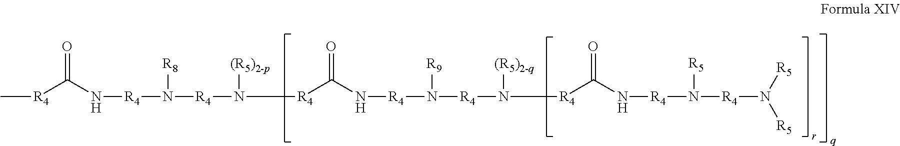

R₈ independently represents an R₅ group or independently represents a group according to the following Formula XV:

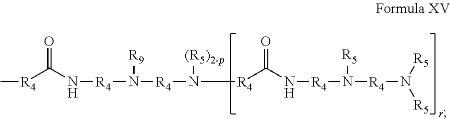

R₉ independently represents an R₅ group or independently represents a group according to the following Formula XVI:

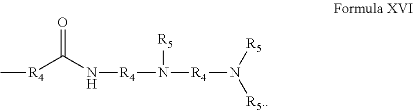

Another aspect of the invention is a pharmaceutical composition comprising one or more amido-amine polymers of the present invention with at least one pharmaceutically acceptable carrier. The amido-amine polymers described herein have several therapeutic applications. For example, the amido-amine polymers are useful in removing compounds or ions such as anions, for example phosphorous-containing compounds or phosphorous containing ions such as organophosphates and/or phosphates, from the gastrointestinal tract, such as from the stomach, small intestine and/or large intestine. In some embodiments, the amido-amine polymers are used in the treatment of phosphate imbalance disorders and renal diseases.

In some embodiments, the invention comprises an amido-amine compound or amido-amine polymer that comprises an amido-amine dendrimer or residue thereof, where the dendrimer comprises a multi-amine core and branches emanating from the core, where the branches may be formed using a reiterative reaction sequence that includes a Michael addition of a substituted or un-substituted α, β unsaturated carboxylic acid or ester and a condensation reaction between the acid or ester group of the substituted or un-substituted α, β unsaturated carboxylic acid or ester with a multi-amine.

In yet another aspect, the amido-amine polymers are useful for removing other solutes, such as chloride, bicarbonate, and/or oxalate containing compounds or ions. Amido-amine polymers removing oxalate compounds or ions find use in the treatment of oxalate imbalance disorders. Amido-amine polymers removing chloride compounds or ions find use in treating acidosis, for example. In some embodiments, the amido-amine polymers are useful for removing bile acids and related compounds.

The invention further provides compositions containing any of the above amido-amine polymers where the amido-amine polymer is in the form of particles and where the particles are encased in one or more shells.

In another aspect, the invention provides pharmaceutical compositions. In one embodiment, the pharmaceutical composition contains an amido-amine polymer of the invention and a pharmaceutically acceptable excipient. In some embodiments, the composition is a liquid formulation in which the amido-amine polymer is dispersed in a liquid vehicle, such as water, and suitable excipients. In some embodiments, the invention provides a pharmaceutical composition comprising an amido-amine polymer for binding a target compound or ion, and one or more suitable pharmaceutical excipients, where the composition is in the form of a tablet, sachet, slurry, food formulation, troche, capsule, elixir, suspension, syrup, wafer, chewing gum or lozenge. In some embodiments the composition contains a pharmaceutical excipient selected from the group consisting of sucrose, mannitol, xylitol, maltodextrin, fructose, sorbitol, and combinations thereof. In some embodiments the target anion of the amido-amine polymer is an organophosphate and/or phosphate. In some embodiments the amido-amine polymer is more than about 50% of the weight of the tablet. In some embodiments, the tablet is of cylindrical shape with a diameter of from about 12 mm to about 28 mm and a height of from about 1 mm to about 8 mm and the amido-amine polymer comprises more than 0.6 to about 2.0 gm of the total weight of the tablet.

In some of the compositions of the invention, the excipients are chosen from the group consisting of sweetening agents, binders, lubricants, and disintegrants. Optionally, the amido-amine polymer is present as particles of less than about 80 μm mean diameter. In some of these embodiments, the sweetening agent is selected from the group consisting of sucrose, mannitol, xylitol, mahodextrin, fructose, and sorbitol, and combinations thereof.

In some embodiments, the invention provides amido-amine compounds, amido-amine polymers or compositions that comprise an amido-amine dendrimer or residue thereof, where the amido-amine dendrimer is formed from a core that comprises a multi-amine that is substituted with one or more groups independently represented by one or both of the following Formulas XVII and Formula XVIII:

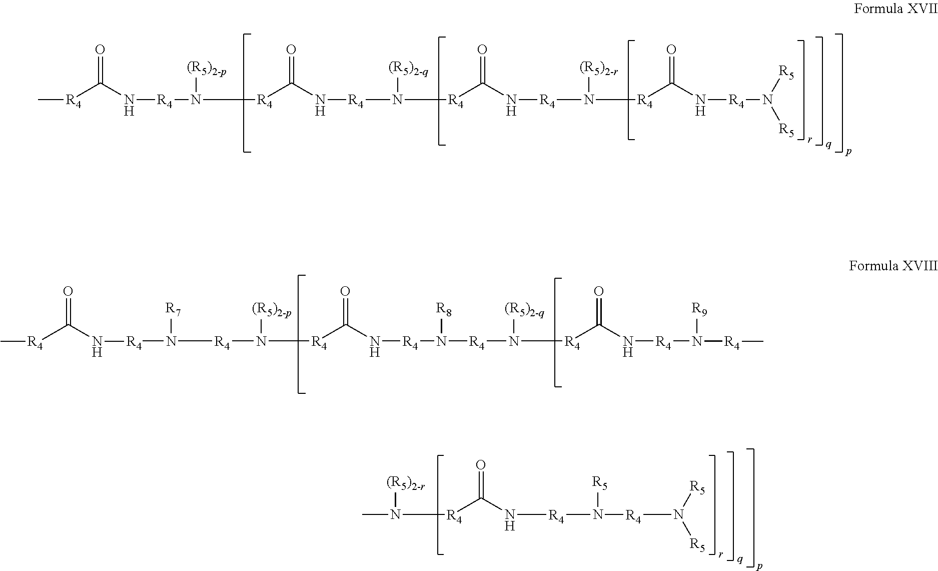

where p, q and r independently represent an integer from 0-2; R₄ independently represents:

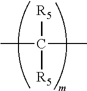

where m independently represents an integer from 1-20; R₅ independently represents a hydrogen radical; a substituted or un-substituted alkyl radical; a substituted or un-substituted aryl radical; or R₅ and a neighboring R₅ together represent a link or links comprising a residue of a crosslinking agent, a substituted or un-substituted alicyclic radical, a substituted or un-substituted aromatic radical, or a substituted or un-substituted heterocyclic radical; or R₅ represents a link with another compound or a residue thereof; R₇ independently represents an R₅ group or independently represents a group according to the following Formula XIV:

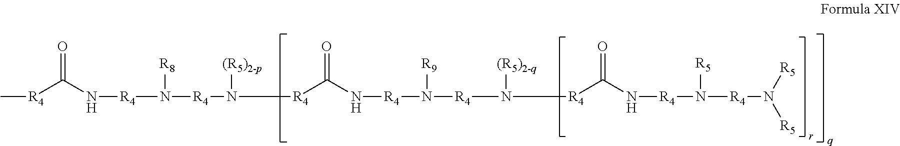

R₈ independently represents an R₅ group or independently represents a group according to the following Formula XV:

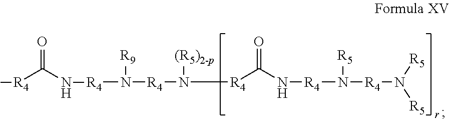

R₉ independently represents an R₅ group or independently represents a group according to the following Formula XVI:

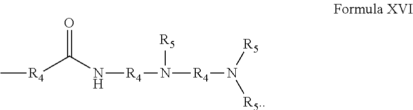

### DETAILED DESCRIPTION OF THE INVENTION

In one aspect, the present invention provides amido-amine compounds, amido-amine polymers, compositions and methods of using amido-amine polymers or compositions comprising an amido-amine polymer or amido-amine compound or residue thereof, where the amido-amine compound is represented by Formula I. In some embodiments, the compositions may comprise amido-amine polymers that may be derived from two or more of the amido-amine compounds described herein.

In addition, some embodiments may include multiple amido-amine compounds or residues thereof that repeat in a copolymer or polymer. Such polymers may include one or more additional compounds that may be included in a polymer backbone or as pendant groups either individually or as repeating groups, and that may provide separation between the individual amido-amine polymers.

As used herein, unless otherwise stated, the term “derived from” is understood to mean: produced or obtained from another substance by chemical reaction, especially directly derived from the reactants, for example an amido-amine compound may be derived from the reaction of a multi-amine monomer and a monofunctional monomer comprising two or more amine-reactive groups. Additionally, an amido-amine compound that is reacted with a linking agent, such as a crosslinking agent results in an amido-amine polymer that is derived from the amido-amine compound and the linking agent.

In some embodiments, the invention is an amido-amine compound, amido-amine dendrimer, amido-amine polymer or composition, or a method for removing a compound or ion, such as a phosphorous-containing compound or a phosphorous-containing ion (e.g. phosphate), from the gastrointestinal tract of an animal by administering an effective amount of an amido-amine polymer that comprises at least one amido-amine compound or residue thereof or at least one amido-amine dendrimer or residue thereof. The amido-amine compound or amido-amine dendrimer may be derived from a multi-amine and a multifunctional compound, where the multifunctional compound comprises two or more amine-reactive groups. In some embodiments, the amine reactive groups are independently selected from the group consisting of vinyl groups, carboxylic acid groups and ester groups and combinations thereof.

In some embodiments, the multifunctional monomer comprising two or more amine-reactive groups is selected from the group consisting of

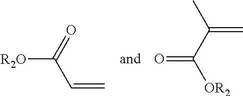

where R₂ independently represents a hydrogen radical or a branched or unbranched, substituted or un-substituted alkyl radical, for example a C₁ to C₂₀ alkyl radical, such as a C₁, C₂, C₃, C₄, C₅ or C₆ radical, such as, for example,

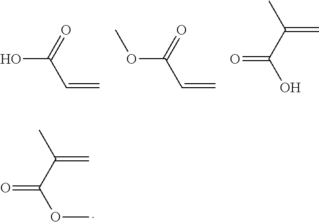

In some embodiments, the multi-amine is selected from the group consisting of:

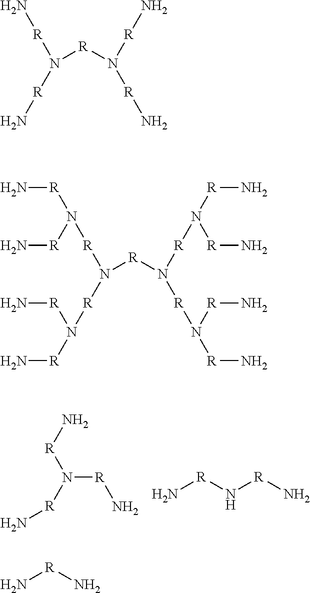

and combinations thereof, wherein R independently represents a branched or unbranched, substituted or un-substituted alkyl radical, for example a C₁ to C₂₀ alkyl radical, such as a C₁, C₂, C₃, C₄, C₅ or C₆ radical, such as, for example,

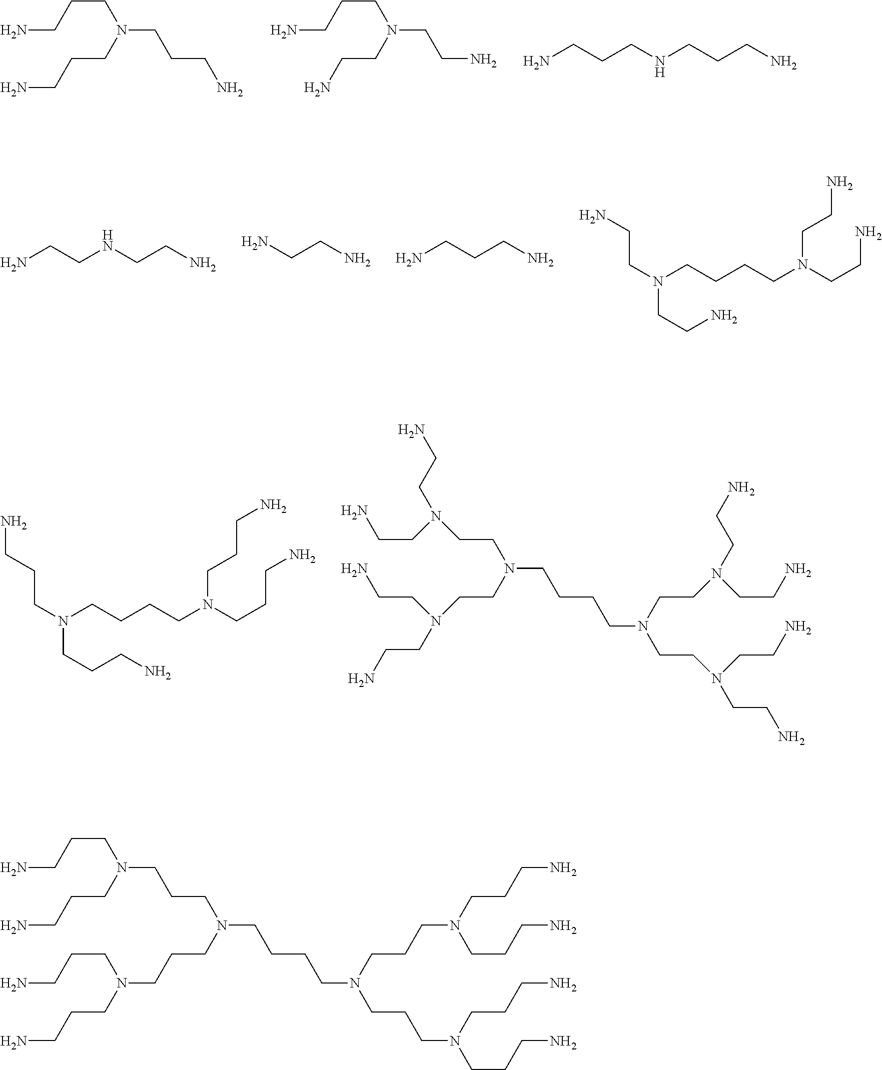

and combinations thereof. In some embodiments, the multi-amines may be a combination of multi-amines such as, for example, combinations as follows:

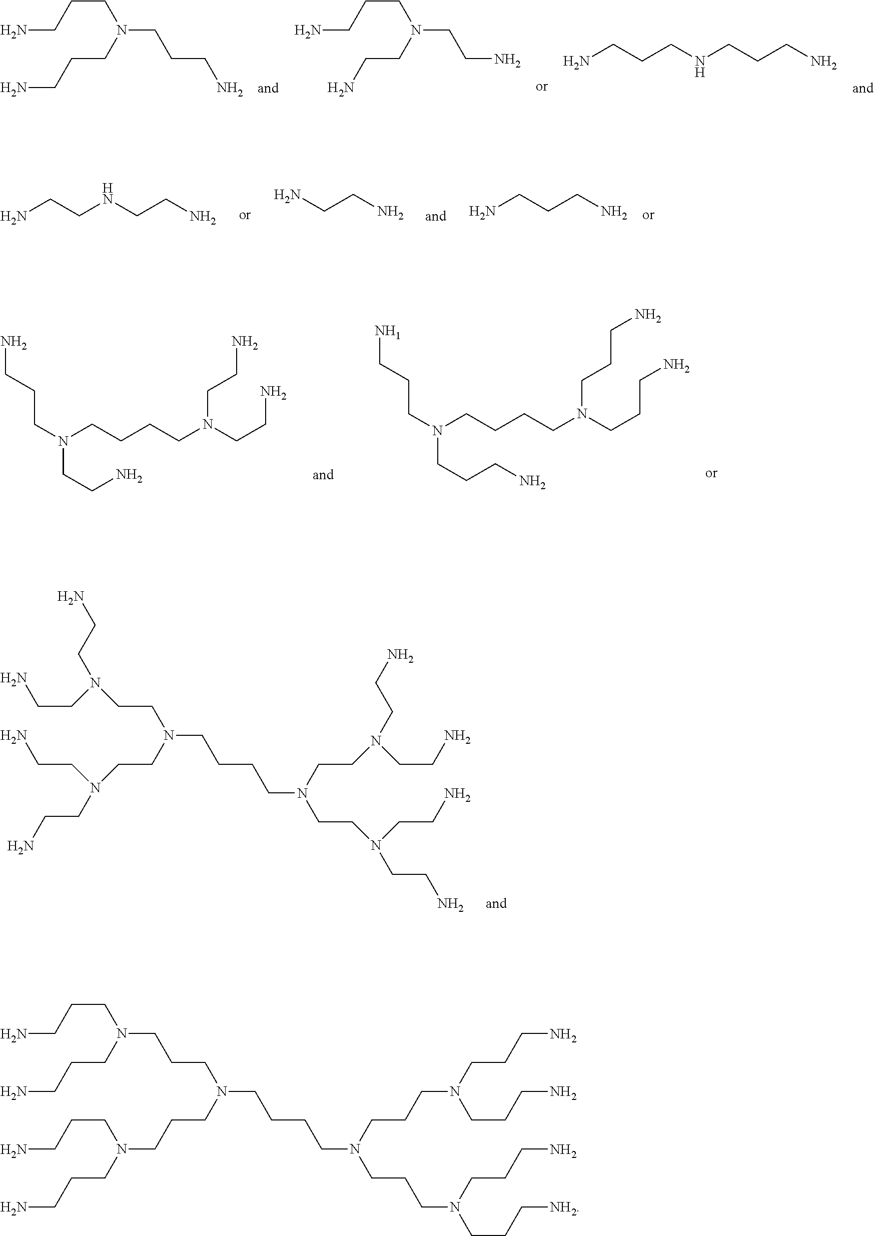

In other embodiments, the multi-amine may be any combination of two or more of any of the multi-amines.

In some embodiments, the invention is an amido-amine compound, amido-amine polymer or composition, or a method for removing a compound or ion, such as a phosphorous-containing compound or a phosphorous-containing ion (e.g. phosphate), from the gastrointestinal tract of an animal by administering an effective amount of an amido-amine polymer that comprises at least one amido-amine compound or residue thereof or at least one amido-amine dendrimer or residue thereof, where the amido-amine compound or amido-amine dendrimer is derived from compounds according to the following Formulas I and II:

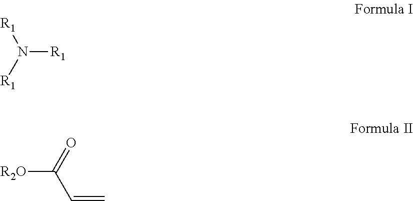

wherein R₁ independently represents a hydrogen radical, —RNH₂, —R—N—(R—NH₂)₂ or —R—N—(R—N—(R—NH₂)₂)₂, wherein R independently represents a branched or unbranched, substituted or un-substituted alkyl radical for example a C₁ to C₂₀ alkyl radical, such as a C₁, C₂, C₃, C₄, C₅ or C₆ radical, with the proviso that at least one R₁ is not a hydrogen radical and R₂ independently represents a hydrogen radical or a branched or unbranched, substituted or un-substituted alkyl radical, for example a C₁ to C₂₀ alkyl radical, such as a C₁, C₂, C₃, C₄, C₅ or C₆ radical.

In some embodiments the compound according to Formula I is selected from the group consisting of:

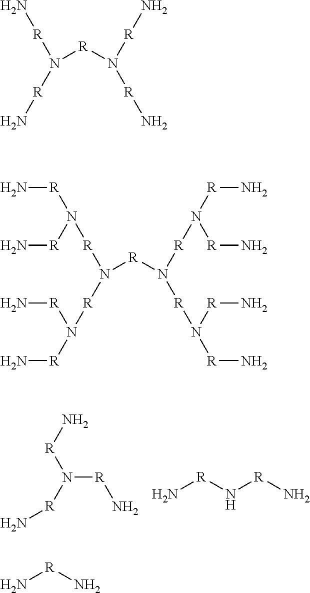

and combinations thereof, wherein R independently represents a branched or unbranched, substituted or un-substituted alkyl radical, for example a C₁ to C₂₀ alkyl radical such as a C₁, C₂, C₃, C₄, C₅ or C₆ radical, such as, for example,

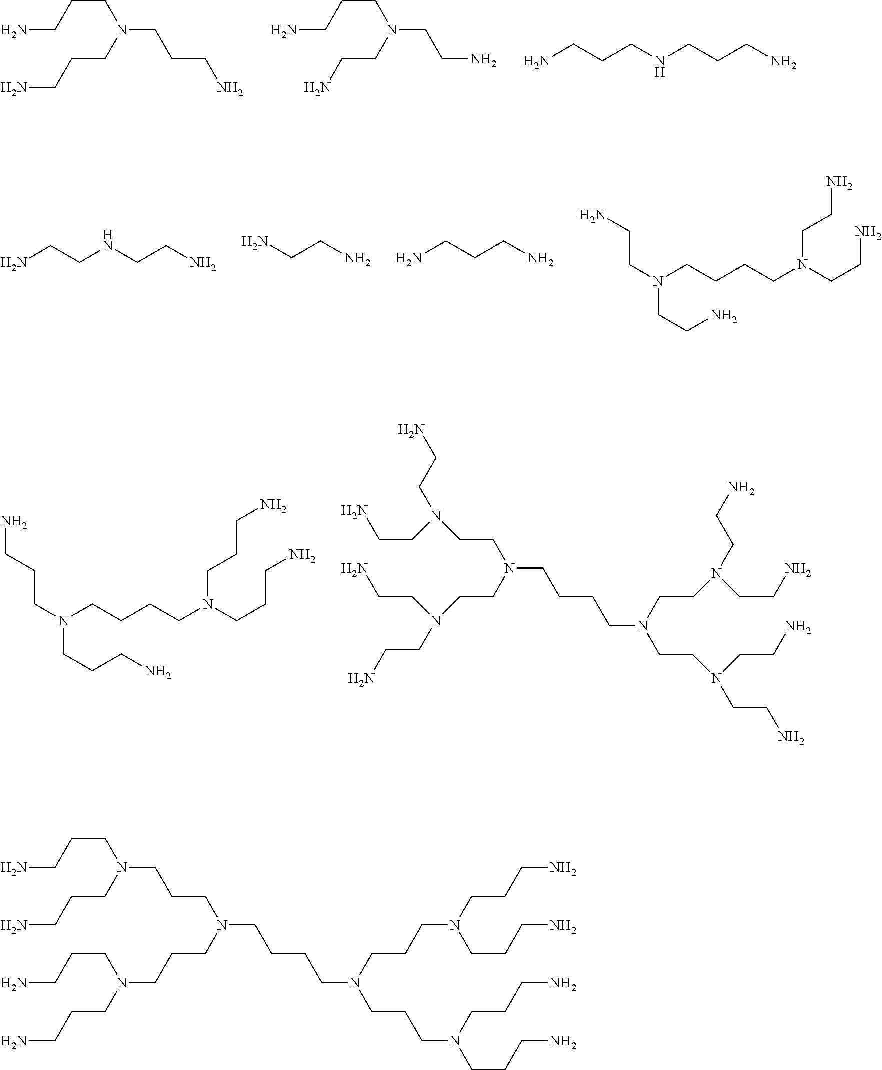

and combinations thereof. In some embodiments, the compound according to Formula I may be a combination of compounds according to Formula I such as, for example, combinations as follows:

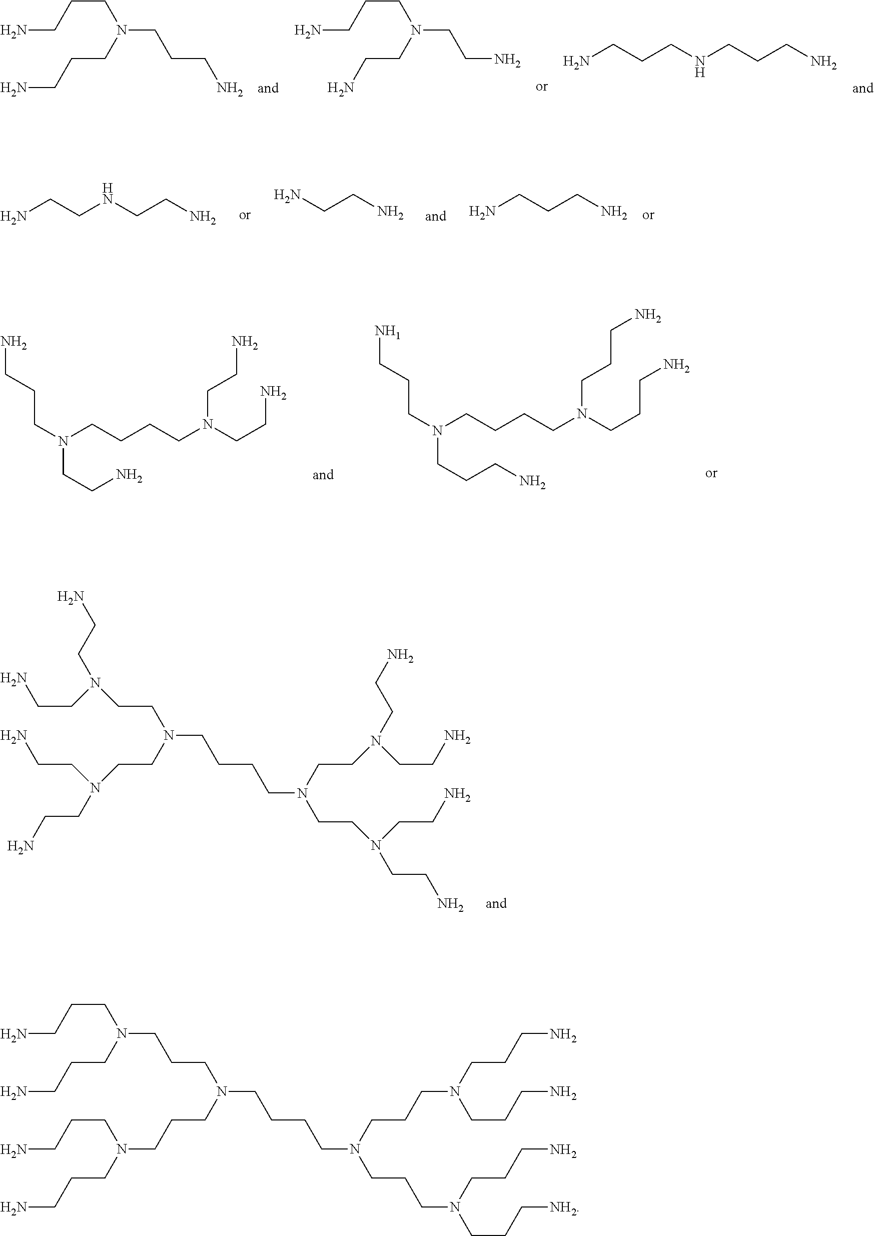

In other embodiments, the compound according to Formula I may be any combination of two or more of any of the compounds according to Formula I.

In some embodiments, the invention is an amido-amine compound, amido-amine polymer or composition, or a method for removing a compound or ion, such as a phosphorous-containing compound or a phosphorous-containing ion (e.g. phosphate), from the gastrointestinal tract of an animal by administering an effective amount of an amido-amine polymer that comprises at least one amido-amine compound or residue thereof or at least one amido-amine dendrimer or residue thereof, where the amido-amine compound or amido-amine dendrimer is represented by the following Formula III:

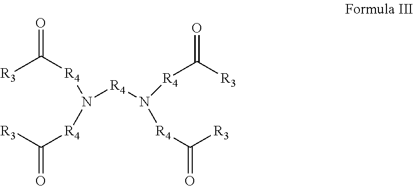

where R₃ independently represents a group represented by the following Formula IV:

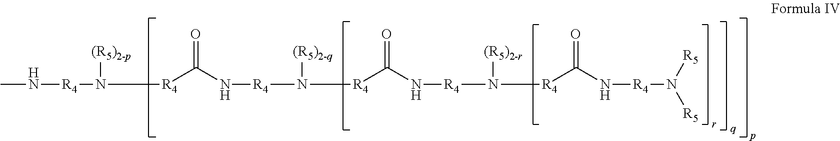

where p, q and r independently represent an integer from 0-2, for example 0, 1 or 2; R₄ independently represents

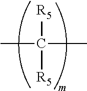

where m independently represents an integer from 1-20, for example, 1-15, 1-2, 3-6, 7-10, 11-15, such as 1, 2, 3, 4, 5, 6, 7, 8, 9, 10, 11, 12, 13, 14, 15, 16, 17, 18, 19 or 20; R₅ independently represents a hydrogen radical; a substituted or un-substituted alkyl radical; a substituted or un-substituted aryl radical; or R₅ and a neighboring R₅ together represent a link or links comprising a residue of a crosslinking agent, a substituted or un-substituted alicyclic radical, a substituted or un-substituted aromatic radical, or a substituted or un-substituted heterocyclic radical; or R₅ represents a link with another compound or a residue thereof. Examples of such compounds include compounds according to Formulas V, VI or VII:

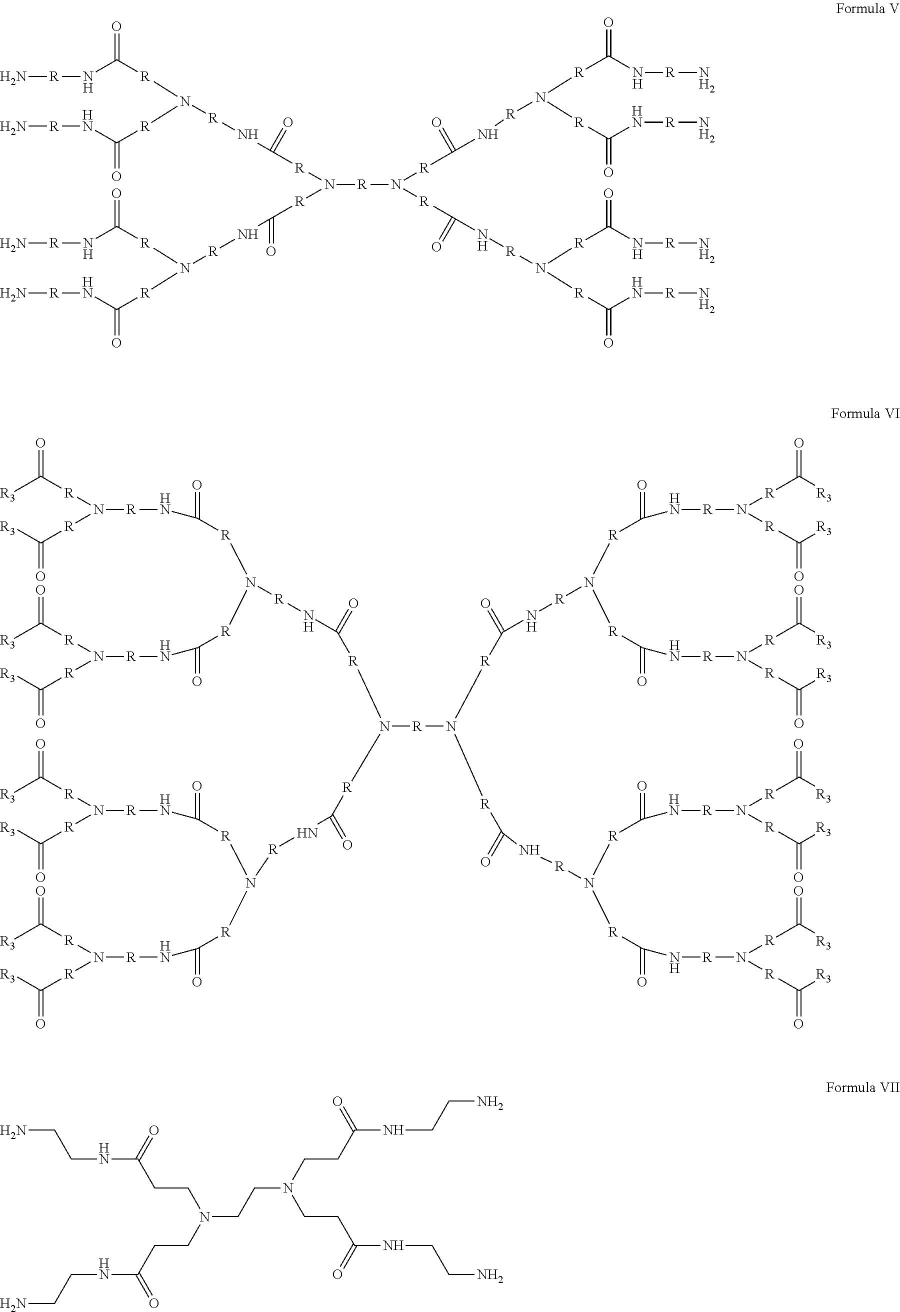

where R independently represents a branched or unbranched, substituted or un-substituted alkyl radical, for example a C₁ to C₂₀ alkyl radical, such as a C₁, C₂, C₃, C₄, C₅ or C₆ radical.

In some embodiments, the invention is an amido-amine compound, amido-amine polymer or composition, or a method for removing a compound or ion, such as a phosphorous-containing compound or a phosphorous-containing ion (e.g. phosphate), from the gastrointestinal tract of an animal by administering an effective amount of an amido-amine polymer that comprises at least one amido-amine compound or residue thereof or at least one amido-amine dendrimer or residue thereof, where the amido-amine compound or amido-amine dendrimer is represented by the following Formula VIII:

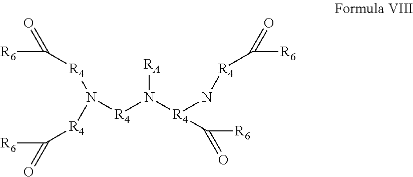

R₆ independently represents a group represented by the following Formula IX:

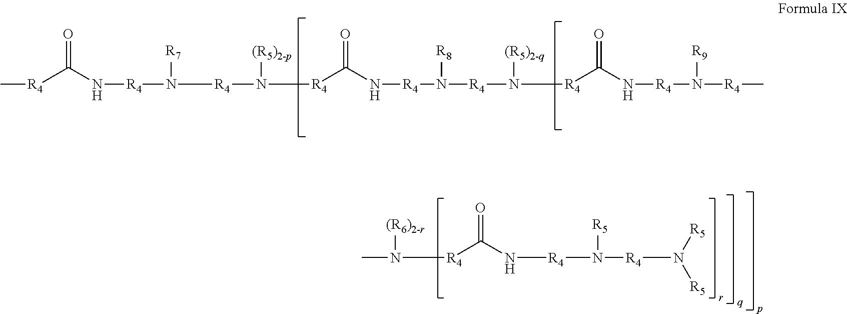

where p, q and r independently represent an integer from 0-2; R₄ independently represents:

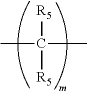

where m independently represents an integer from 1-20, for example, 1-15, 1-2, 3-6, 7-10, 11-15, such as 1, 2, 3, 4, 5, 6, 7, 8, 9, 10, 11, 12, 13, 14, 15, 16, 17, 18, 19 or 20; R₅ independently represents a hydrogen radical; a substituted or un-substituted alkyl radical; a substituted or un-substituted aryl radical; or R₅ and a neighboring R₅ together represent a link or links comprising a residue of a crosslinking agent, a substituted or un-substituted alicyclic radical, a substituted or un-substituted aromatic radical, or a substituted or un-substituted heterocyclic radical; or R₅ represents a link with another compound or a residue thereof; R_{A} independently represents an R₅ group or a —R₄—CO—R₆ group; R₇ independently represents an R₅ group or independently represents a group according to the following Formula XIV:

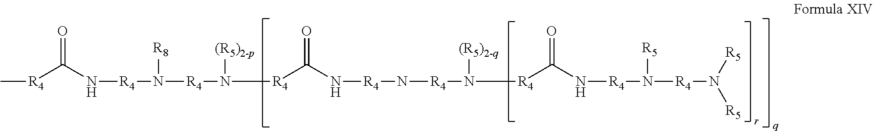

R₈ independently represents an R₅ group or independently represents a group according to the following Formula XV:

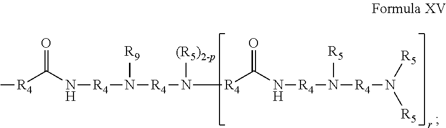

R₉ independently represents an R₅ group or independently represents a group according to the following Formula XVI:

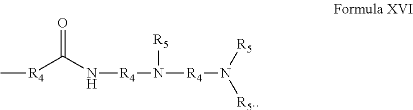

Examples of such compounds include, for example, compounds represented by the following Formula X:

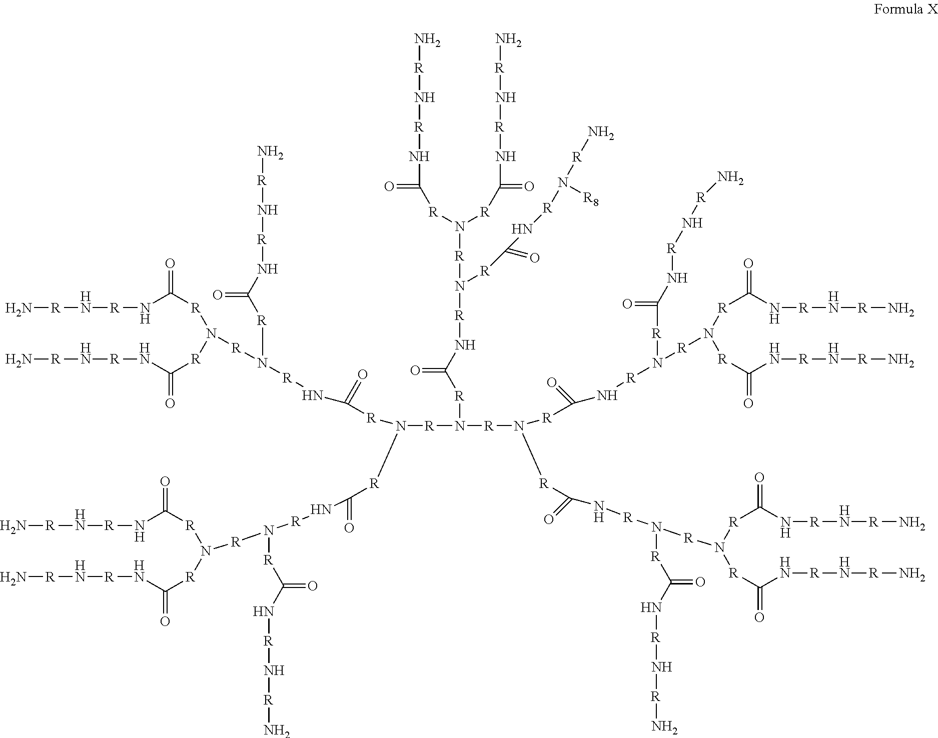

where R independently represents a branched or unbranched, substituted or un-substituted alkyl radical, for example a C₁ to C₂₀ alkyl radical, such as a C₁, C₂, C₃, C₁, C₅ or C₆ radical.

In some embodiments, the invention is an amido-amine compound, amido-amine polymer or composition, or a method for removing a compound or ion, such as a phosphorous-containing compound or a phosphorous-containing ion (e.g. phosphate), from the gastrointestinal tract of an animal by administering an effective amount of an amido-amine polymer that comprises at least one amido-amine compound or residue thereof or at least one amido-amine dendrimer or residue thereof, where the amido-amine compound or amido-amine dendrimer is derived from compounds according to the following Formulas II and XI:

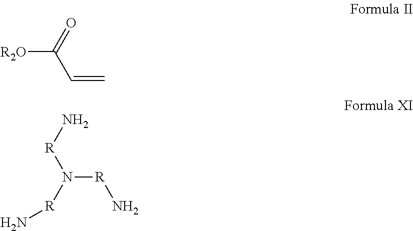

wherein R independently represents a branched or unbranched, substituted or un-substituted alkyl radical, for example a C₁ to C₂₀ alkyl radical, such as a C₁, C₂, C₃, C₄, C₅ or C₆ radical; R₂ independently represents a hydrogen radical or a branched or unbranched, substituted or un-substituted alkyl radical, for example a C₁ to C₂₀ alkyl radical, such as a C₁, C₂, C₃, C₄, C₅ or C₆ radical.

In some embodiments, the compound according to Formula XI comprises:

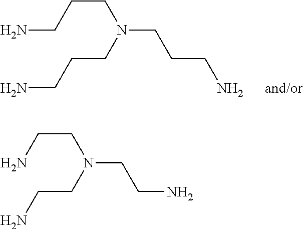 [SMILES: NCCCN(CCCN)CCCN ; NCCN(CCN)CCN]

In some embodiments, the invention is an amido-amine compound, amido-amine polymer or composition, or a method for removing a compound or ion, such as a phosphorous-containing compound or a phosphorous-containing ion (e.g. phosphate), from the gastrointestinal tract of an animal by administering an effective amount of an amido-amine polymer that comprises at least one amido-amine compound or residue thereof or at least one amido-amine dendrimer or residue thereof, where the amido-amine compound or amido-amine dendrimer is derived from compounds according to the following Formulas II and XII:

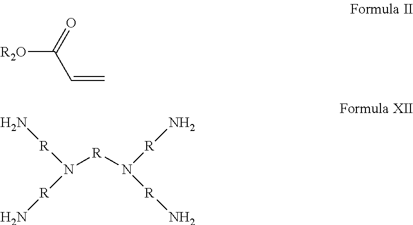

wherein R independently represents a branched or unbranched, substituted or un-substituted alkyl radical, for example a C₁ to C₂₀ alkyl radical, such as a C₁, C₂, C₃, C₄, C₅ or C₆ radical; R₂ independently represents a hydrogen radical or a branched or unbranched, substituted or un-substituted alkyl radical, for example a C₁ to C₂₀ alkyl radical, such as a C₁, C₂, C₃, C₄, C₅ or C₆ radical.

In some embodiments, the compound according to Formula XII comprises:

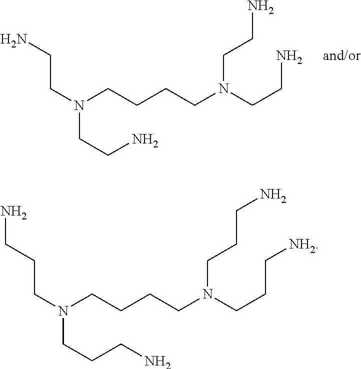 [SMILES: NCCN(CCN)CCCCN(CCN)CCN ; NCCCN(CCCN)CCCCN(CCCN)CCCN]

In some embodiments, the invention is an amido-amine compound, amido-amine polymer or composition, or a method for removing a compound or ion, such as a phosphorous-containing compound or a phosphorous-containing ion (e.g. phosphate), from the gastrointestinal tract of an animal by administering an effective amount of an amido-amine polymer that comprises at least one amido-amine compound or residue thereof or at least one amido-amine dendrimer or residue thereof, where the amido-amine compound or amido-amine dendrimer is derived from compounds according to the following Formulas II and XIII:

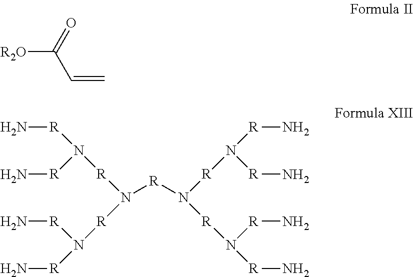

wherein R independently represents a branched or unbranched, substituted or un-substituted alkyl radical, for example a C₁ to C₂₀ alkyl radical, such as a C₁, C₂, C₃, C₄, C₅ or C₆ radical; R₂ independently represents a hydrogen radical or a branched or unbranched, substituted or un-substituted alkyl radical, for example a C₁ to C₂₀ alkyl radical, such as a C₁, C₂, C₃, C₄, C₅ or C₆ radical.

In some embodiments, the compound according to Formula XII comprises:

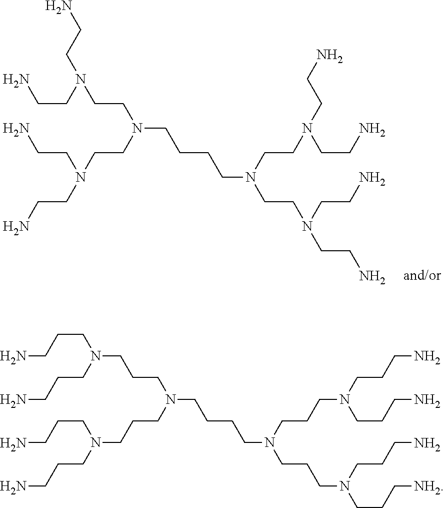 [SMILES: NCCN(CCN)CCN(CCCCN(CCN(CCN)CCN)CCN(CCN)CCN)CCN(CCN)CCN ; NCCCN(CCCN)CCCN(CCCCN(CCCN(CCCN)CCCN)CCCN(CCCN)CCCN)CCCN(CCCN)CCCN]

In some embodiments, the invention is an amido-amine compound, amido-amine polymer or composition, or a method for removing a compound or ion, such as a phosphorous-containing compound or a phosphorous-containing ion (e.g. phosphate) from the gastrointestinal tract of an animal by administering an effective amount of an amido-amine polymer that comprises at least one amido-amine compound or residue thereof, where the amido-amine compound comprises an amido-amine dendrimer or residue thereof, the dendrimer having a core that is a residue of one or more multi-amine compounds and a residue of one or more substituted or un-substituted α, β unsaturated carboxylic acids or esters.

In some embodiments, dendrimers of the present invention may be formed from any suitable reaction scheme. Dendrimers are macromolecular compounds that comprise a core that includes functional groups and dendritic branches that may be formed through a series of iterative reaction sequences with the functional groups on the core to form a branched macromolecule. In some embodiments the reactive functional groups comprise hydroxyl groups and/or amine groups. The functional groups will have functionalities that are dependent on the type of group. For example, hydroxyl groups have a functionality of one, while primary amines generally have a functionality of 2, though they may be quaternized. In some embodiments, an amido-amine polymer comprises a dendrimer or residue thereof where the dendrimer comprises a multi-amine core that comprises a residue of one or more amine groups and a residue of one or more substituted or un-substituted α, β unsaturated carboxylic acid or ester groups, the amido-amine polymer further comprising a crosslinking or other linking agent or residue thereof. Some examples of substituted or un-substituted α, β unsaturated carboxylic acids or esters include acrylic acid, methyl acrylate, methacrylic acid and methyl methacrylate.

In some embodiments, dendrimers of the present invention are prepared by a Michael addition of a substituted or un-substituted α, β unsaturated carboxylic acid or ester to one or more of the amine groups on a multi-amine core to replace the hydrogens of the amine group with a carboxylic acid or ester group resulting in amine linkages to the core via the nitrogen atom of the amine group. The ester or acid groups of the resulting compound are then condensed with the same or a different multi-amine by replacing the hydroxyl or alkoxy group of the carboxylic acid or ester group with an amine group from the multi-amine resulting in an amide linkage. The Michael addition and subsequent condensation may be repeated on the remaining amine groups of the multi amine generally yielding a branched tertiary amido-amine. Subsequent Michael additions and condensations may be repeated one or more times to provide the branched structure characteristic of dendrimers. While a schematic of this process is provided below in Scheme I, using methyl acrylate as the substituted or un-substituted α, β unsaturated carboxylic acid or ester and diaminopropane as the multi-amine, it should be noted that any multi-amines described herein and any substituted or un-substituted α, β unsaturated carboxylic acid or ester described herein may be used:

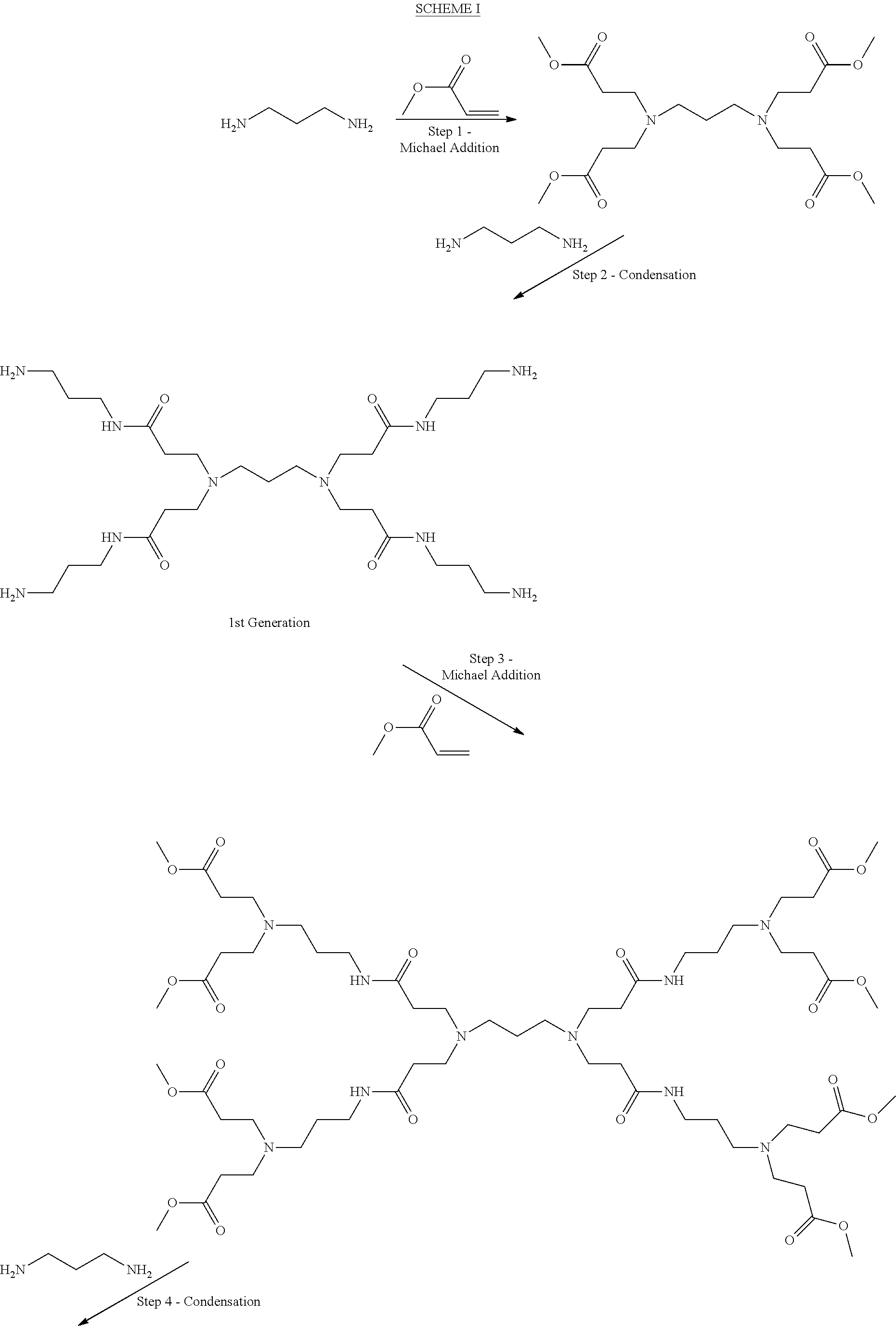

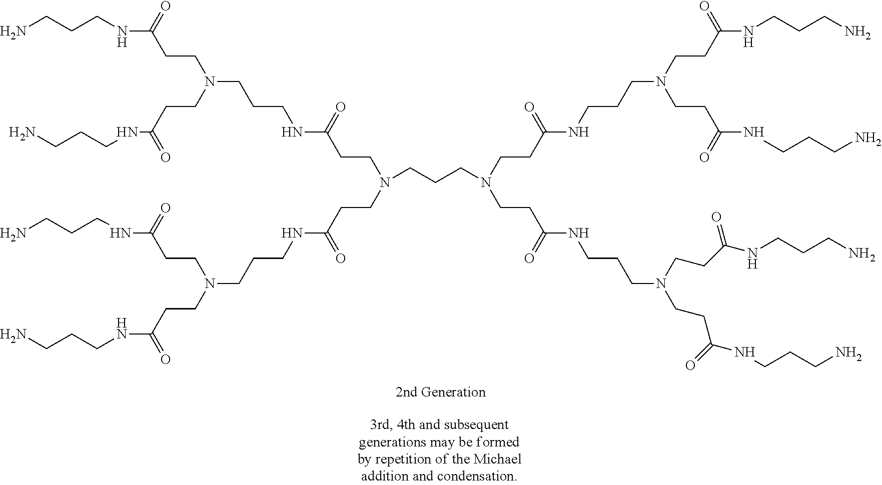 [SMILES: NCCCNC(=O)CCN(CCCNC(=O)CCN(CCCN(CCC(=O)NCCCN(CCC(=O)NCCCN)CCC(=O)NCCCN)CCC(=O)NCCCN(CCC(=O)NCCCN)CCC(=O)NCCCN)CCC(=O)NCCCN(CCC(=O)NCCCN)CCC(=O)NCCCN)CCC(=O)NCCCN]

In some embodiments, each iteration of Michael addition and subsequent condensation may be considered one generation. Thus, for some embodiments, a compound having one generation of dendritic branching may have undergone one iteration of Michael addition and condensation, compounds having two generations of dendritic branching may have undergone two iterations of Michael addition and condensation, compounds having three generations of dendritic branching may have undergone three iterations of Michael addition and condensation, compounds having four generations of dendritic branching may have undergone four iterations of Michael addition and condensation, etc. Generally dendrimers according to some embodiments of the present invention may have from 1-10, such as 2, 3, 4, 5, 6, 7, 8, or 9 generations of dendritic branching.

Scheme I shows multiple iterations of Michael addition and subsequent condensation using the same multi-amine or compound according to Formula I. Any multi-amine or compound according to Formula I may be used in any of the appropriate steps. For example, in some embodiments, the invention includes use of different multi-amines for different condensation steps. For example, each separate condensation step may include a different multi-amine or compound according to Formula I. Alternatively, the steps may include patterns of use of different multi-amines for the condensation steps including alternating the multi-amines or compounds according to Formula I and any other pattern. For example, in some embodiments, the multi-amine in Step 1 may be

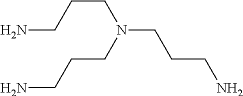 [SMILES: NCCCN(CCCN)CCCN]

and the multi-amine in Step three may be

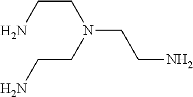

and then for step five the first multi-amine may be used again and the multi-amine used may subsequently alternate in this pattern. Any of the multi-amines or compounds according to Formula I may be used in any combination.

In some embodiments, a method of making an amido-amine polymer comprises reacting a multi-amine core with a substituted or un-substituted α, β unsaturated carboxylic acid or ester using a Michael addition reaction to form a polyacid, condensing the polyacid with the same or a different multi-amine to form a primary amine, repeating the Michael addition and condensation on the primary amine one or more times to form an amido-amine dendrimer; and crosslinking the amido-amine dendrimer with a crosslinking agent.

Some embodiments of the invention may comprise a polymer or composition or a method for removing a compound or ion, such as a phosphorous-containing compound or a phosphorous-containing ion (e.g. phosphate) from the gastrointestinal tract of an animal by administering an effective amount of a polymer that comprises two or more amido-amine compounds or residues thereof or amido-amine dendrimers or residues thereof, that comprise a residue of two or more multi-amines, a residue of one or more substituted or un-substituted α, β unsaturated carboxylic acids or esters and a residue of one or more crosslinking or other linking agents. In some embodiments, the polymer network may include one or more amido-amine dendrimers or residues thereof.

In some embodiments, the invention is a method for reducing blood phosphate levels by 5-100%, such as 10%, 20%, 30%, 40%, 50%, 60%, 70%, 80%, or 90% in a patient in need thereof, the method comprising administering a therapeutically effective amount of an amido-amine polymer or composition according to the invention to the patient. In some embodiments, the invention is a method for reducing urinary phosphorous by 5-100%, such as 10%, 20%, 30%, 40%, 50%, 60%, 70%, 80%, or 90% in a patient in need thereof, the method comprising administering a therapeutically effective amount of an amido-amine polymer or composition according to the invention to the patient.

In some embodiments, the invention is a method of treating a phosphate imbalance disorder such as hyperphosphatemia comprising administering a therapeutically effective amount of one or more polymers or copolymers of the invention or a composition comprising one or more one or more polymers or copolymers of the invention to a patient in need thereof.

In some embodiments, the composition includes a mixture of more than one polymer or copolymer of the invention, for example 2-20 such as 2, 3, 4, 5, 6, 7, 8, 9 or 10 polymers or copolymers of the invention.

In some embodiments, the invention comprises a polymer or copolymer of the invention derived from a mixture of multi-amine compounds, a pharmaceutical composition comprising such a polymer or copolymer, or a method of using the same in a therapeutically effective amount to remove a compound or ion, such as a phosphorous-containing compound or a phosphorous-containing ion (e.g. phosphate), from the gastrointestinal tract of an animal.

Other embodiments of the invention include pendant amido-amine polymers formed with amido-amine compounds or residues thereof as pendant groups on a polymer or polymerized backbone of a polymer. Such pendant amido-amine polymers may be formed by adding one or more polymerizable groups to one or more amine groups on an amido-amine compound to form an amido-amine monomer and then subsequently polymerizing the polymerizable group to form a pendant amido-amine polymer comprising an amido-amine compound or residue thereof. A schematic example of such an addition follows [it should be noted in the following that an amido-amine compound designated as “AC” is intended to represent an amido-amine compound or residue thereof, of the invention, with one of its amine groups depicted for purposes of illustrating how a polymerizable group may be added to an amido-amine compound]:

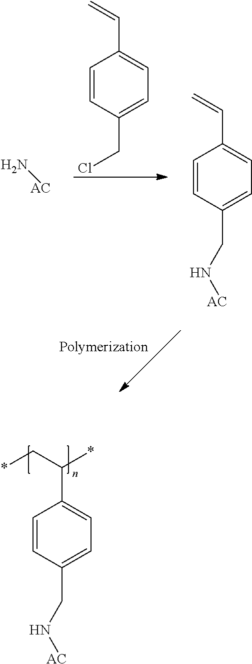

Non-limiting examples of other polymerizable groups that may be used with amido-amine compounds or residues thereof according to embodiments of the invention include:

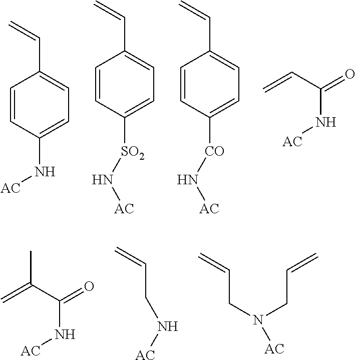

One or more polymerizable groups may be added to each amido-amine compound and thus it is possible to have mixtures of amido-amine monomers having various pendant ACs having differing numbers of polymerizable groups. In addition, the pendant amido-amine polymers made in this fashion may be modified, crosslinked, formed into a network or substituted post polymerization. Such modification may be performed for any number of reasons, including to improve efficacy, tolerability or reduce side effects.

Amido-amine monomers may also be formed by addition of amido-amine compounds to amine-reactive polymers by reacting one or more amine groups of the amido-amine monomers with one or amine-reactive groups on the amine-reactive polymers. Examples of some amine reactive polymers include:

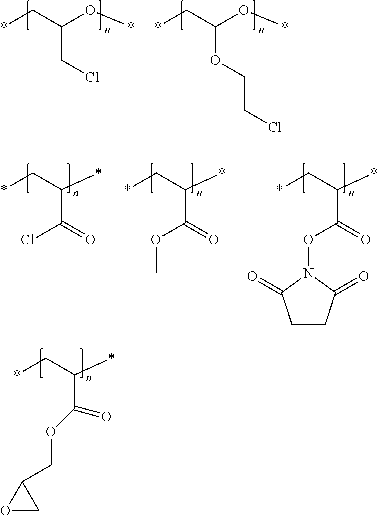

The amido-amine compounds or amido-amine monomers may also serve as multifunctional amido-amine monomers to form polymers. For example, when the amido-amine compounds or the polymers formed from the amido-amine monomers are crosslinked, the crosslinking reaction may be carried out either in solution of bulk (i.e. using the neat amido-amine and neat crosslinking agents) or in dispersed media. When a bulk process is used, solvents are selected so that they co-dissolve the reactants and do not interfere with the crosslinking reaction. Suitable solvents include water, low boiling alcohols (methanol, ethanol, butanol), dimethylformamide, dimethylsulfoxide, acetone, methylethylketone, and the like.

Other polymerization methods may include a single polymerization reaction, stepwise addition of individual monomers via a series of reactions, the stepwise addition of blocks of monomers, combinations of the foregoing, or any other method of polymerization, such as, for example, direct or inverse suspension, condensation, emulsion, precipitation techniques, polymerization in aerosol or using bulk polymerization/crosslinking methods and size reduction processes such as extrusion and grinding. Processes can be carried out as batch, semi-continuous and continuous processes. For processes in dispersed media, the continuous phase can be selected from apolar solvents such as toluene, benzene, hydrocarbon, halogenated solvents, supercritical carbon dioxide, and the like. With a direct suspension process, water can be used, although salt brines are also useful to “salt out” the amido-amine and crosslinking agents in a droplet separate phase.

Amido-amine compounds and amido-amine monomers of the invention may be copolymerized with one or more other monomers or oligomers or other polymerizable groups, may be crosslinked, may have crosslinking or other linking agents or monomers within the polymer backbone or as pendant groups or may be formed or polymerized to form a polymer network or mixed polymer network comprising: amido-amine compounds or residues thereof, amido-amine monomers or residues thereof, crosslinking agents or residues thereof, or other linking agents or residues thereof. The network may include multiple connections between the same or different molecules that may be direct or may include one or more linking groups such as crosslinking agents or other linking agents such as monomers or oligomers or residues thereof.

Non-limiting examples of comonomers which may be used alone or in combination include: styrene, substituted styrene, alkyl acrylate, substituted alkyl acrylate, alkyl methacrylate, substituted alkyl methacrylate, acrylonitrile, methacrylonitrile, acrylamide, methacrylamide, N-alkylacrylamide, N-alkylmethacrylamide, N,N-dialkylacrylamide, N,N-dialkylmethacrylamide, isoprene, butadiene, ethylene, vinyl acetate, N-vinyl amide, maleic acid derivatives, vinyl ether, allyle, methallyl monomers and combinations thereof. Functionalized versions of these monomers may also be used. Additional specific monomers or comonomers that may be used in this invention include, but are not limited to, methyl methacrylate, ethyl methacrylate, propyl methacrylate (all isomers), butyl methacrylate (all isomers), 2-ethylhexyl methacrylate, isobornyl methacrylate, methacrylic acid, benzyl methacrylate, phenyl methacrylate, methacrylonitrile, α-methylstyrene, methyl acrylate, ethyl acrylate, propyl acrylate (all isomers), butyl acrylate (all isomers), 2-ethylhexyl acrylate, isobornyl acrylate, acrylic acid, benzyl acrylate, phenyl acrylate, acrylonitrile, styrene, glycidyl methacrylate, 2-hydroxyethyl methacrylate, hydroxypropyl methacrylate (all isomers), hydroxybutyl methacrylate (all isomers), N,N-dimethylaminoethyl methacrylate, N,N-diethylaminoethyl methacrylate, triethyleneglycol methacrylate, itaconic anhydride, itaconic acid, glycidyl acrylate, 2-hydroxyethyl acrylate, hydroxypropyl acrylate (all isomers), hydroxybutyl acrylate (all isomers), N,N-dimethylaminoethyl acrylate, N,N-diethylaminoethyl acrylate, triethyleneglycol acrylate, methacrylamide, N-methylacrylamide, N,N-dimethylacrylamide, N-tert-butylmethacrylamide, N—N-butylmethacrylamide, N-methylolmethacrylamide, N-ethylolmethacrylamide, N-tert-butylacrylamide, N—N-butylacrylamide, N-methylolacrylamide, N-ethylolacrylamide, 4-acryloylmorpholine, vinyl benzoic acid (all isomers), diethylaminostyrene (all isomers), α-methylvinyl benzoic acid (all isomers), diethylamino α-methylstyrene (all isomers), p-vinylbenzene sulfonic acid, p-vinylbenzene sulfonic sodium salt, trimethoxysilylpropyl methacrylate, triethoxysilylpropyl methacrylate, tributoxysilylpropyl methacrylate, dimethoxymethylsilylpropyl methacrylate, diethoxymethylsilylpropyl methacrylate, dibutoxymethylsilylpropyl methacrylate, diisopropoxymethylsilylpropyl methacrylate, dimethoxysilylpropyl methacrylate, diethoxysilylpropyl methacrylate, dibutoxysilylpropyl methacrylate, diisopropoxysilylpropyl methacrylate, trimethoxysilylpropyl acrylate, triethoxysilylpropyl acrylate, tributoxysilylpropyl acrylate, dimethoxymethylsilylpropyl acrylate, diethoxymethylsilylpropyl acrylate, dibutoxymethylsilylpropyl acrylate, diisopropoxymethylsilylpropyl acrylate, dimethoxysilylpropyl acrylate, diethoxysilylpropyl acrylate, dibutoxysilylpropyl acrylate, diisopropoxysilylpropyl acrylate, maleic anhydride, N-phenylmaleimide, N-butylmaleimide, N-vinylformamide, N-vinyl acetamide, allylamine, methallylamine, allylalcohol, methyl-vinylether, ethylvinylether, butylvinyltether, butadiene, isoprene, chloroprene, ethylene, vinyl acetate and combinations thereof.

In some embodiments, amido-amine polymers of the invention are crosslinked using crosslinking agents, and may not dissolve in solvents, and, at most, swell in solvents The swelling ratio may be measured according to the procedure in the Test Methods section below and is typically in the range of about 1 to about 150, such as 1 to about 100, 1 to about 80, 1 to about 60, 1 to about 40, or 1 to about 20; for example 2 to 10, 2.5 to 8, 3 to 6 or less than 5, less than 6, less than 7, less than 10, less than 15 or less than 20. In some embodiments, the amido-amine polymers may include crosslinking or other linking agents that may result in amido-amine polymers that do not form gels in solvents and may be soluble or partially soluble in some solvents.

Crosslinking agents are typically compounds having at least two functional groups that are selected from a halogen group, carbonyl group, epoxy group, ester group, acid anhydride group, acid halide group, isocyanate group, vinyl group, and chloroformate group. The crosslinking agent may be attached to the carbon backbone or to a nitrogen of an amido-amine compound, amido-amine monomer or residue thereof.

Examples of crosslinking agents that are suitable for synthesis of the polymers or dendrimers of the present invention include, but are not limited to, one or more multifunctional crosslinking agents such as: dihaloalkanes, haloalkyloxiranes, alkyloxirane sulfonates, di(haloalkyl)amines, tri(haloalkyl)amines, diepoxides, triepoxides, tetraepoxides, bis(halomethyl)benzenes, tri(halomethyl)benzenes, tetra(halomethyl)benzenes, epihalohydrins such as epichlorohydrin and epibromohydrin poly(epichlorohydrin), (iodomethyl)oxirane, glycidyl tosylate, glycidyl 3-nitrobenzenesulfonate, 4-tosyloxy-1,2-epoxybutane, bromo-1,2-epoxybutane, 1,2-dibromoethane, 1,3-dichloropropane, 1,2-dichloroethane, 1-bromo-2-chloroethane, 1,3-dibromopropane, bis(2-chloroethyl)amine, tris(2-chloroethyl)amine, and bis(2-chloroethyl)methylamine, 1,3-butadiene diepoxide, 1,5-hexadiene diepoxide, diglycidyl ether, 1,2,7,8-diepoxyoctane, 1,2,9,10-diepoxydecane, ethylene glycol diglycidyl ether, propylene glycol diglycidyl ether, 1,4-butanediol diglycidyl ether, 1,2 ethanedioldiglycidyl ether, glycerol diglycidyl ether, 1,3-diglycidyl glyceryl ether, N,N-diglycidylaniline, neopentyl glycol diglycidyl ether, diethylene glycol diglycidyl ether, 1,4-bis(glycidyloxy)benzene, resorcinol digylcidyl ether, 1,6-hexanediol diglycidyl ether, trimethylolpropane diglycidyl ether, 1,4-cyclohexanedimethanol diglycidyl ether, 1,3-bis-(2,3-epoxypropyloxy)-2-(2,3-dihydroxypropyloxy)propane, 1,2-cyclohexanedicarboxylic acid diglycidyl ester, 2,2′-bis(glycidyloxy)diphenylmethane, bisphenol F diglycidyl ether, 1,4-bis(2′,3′-epoxypropyl)perfluoro-n-butane, 2,6-di(oxiran-2-ylmethyl)-1,2,3,5,6,7-hexahydropyrrolo[3,4-f]isoindol-1,3,5,7-tetraone, bisphenol A diglycidyl ether, ethyl 5-hydroxy-6,8-di(oxiran-2-ylmethyl)-4-oxo-4h-chromene-2-carboxylate, bis[4-(2,3-epoxy-propylthio)phenyl]-sulfide, 1,3-bis(3-glycidoxypropyl)tetramethyldisiloxane, 9,9-bis[4-(glycidyloxy)phenyl]fluorine, triepoxyisocyanurate, glycerol triglycidyl ether, N,N-diglycidlycidyl-4-glycidyloxyaniline, isocyanuric acid (S,S,S)-triglycidyl ester, isocyanuric acid (R,R,R)-triglycidyl ester, triglycidyl isocyanurate, trimethylolpropane triglycidyl ether, glycerol propoxylate triglycidyl ether, triphenylolmethane triglycidyl ether, 3,7,14-tris[[3-(epoxypropoxy)propyl]dimethylsilyloxy]-1,3,5,7,9,11,14-heptacyclopentyltricyclo[7.3.3.15, I]heptasiloxane, 4,4′-methylenebis(N,N-diglycidylaniline), bis(halomethyl)benzene, bis(halomethyl)biphenyl and bis(halomethyl)naphthalene, toluene diisocyanate, acrylol chloride, methyl acrylate, ethylene bisacrylamide, pyrometallic dianhydride, succinyl dichloride, dimethylsuccinate. When the crosslinking agent is an alkylhalide compound, a base can be used to scavenge the acid formed during the reaction. Inorganic or organic bases are suitable. NaOH is preferred. The base to crosslinking agent ratio is preferably between about 0.5 to about 2.

In some embodiments, the crosslinking agents may be introduced into the polymerization reaction in an amount of from 0.5 to 25 wt. % based on the total weight of the amido-amine compound or amido-amine monomer, such as from about 2 to about 15 wt. %, from about 2 to about 12 wt. %, from about 3 to about 10 wt. %, or from about 3 to about 6 wt. %, such as 2, 3, 4, 5, 6 wt %. The amount of crosslinking agent necessary may depend on the extent of branching within the amido-amine compound.

In some embodiments the weight averaged molecular weight of the polymers and copolymers, may be typically at least about 1000. For example, the molecular weight may be from about 1000 to about 1,000,000, such as about 2000 to about 750,000, about 3000 to about 500,000, about 5000 to about 250,000, about 10000 to about 100,000, such as from 15,000-80,000, 20,000 to 75,000, 25,000 to 60,000, 30,000 to 50,000, or 40,000 to 45,000.

In some embodiments, the pharmaceutical composition of the present invention comprises an amido-amine polymer comprising at least one amido-amine compound or residue thereof or at least one amido-amine dendrimer or residue thereof, where the amido-amine compound or amido-amine dendrimer is represented by Formula III where R₆ independently represents an H radical or alkyl radical, q and r are 0 and p is 2, m independently represents an integer from 3-6, such as 3, 4, 5 or 6; and 2-6 wt. % crosslinking agent or residue thereof, such as 2 wt. %, 3 wt. %, 4 wt. %, 5 wt. % or 6 wt. % crosslinking agent, where the crosslinking agent is epichlorohydrin, poly(epichlorohydrin), 1,2-dibromoethane, tris(2-chloroethyl)amine or 1,4-butanediol diglycidyl ether. Another pharmaceutical composition embodiment of the present invention comprises an amido-amine polymer comprising at least one amido-amine compound or residue thereof or an at least one amido-amine dendrimer or residue thereof, where the amido-amine compound or amido-amine dendrimer is represented by Formula III where R₆ independently represents a H radical or alkyl radical, q is 0 and r and p both are 2, m independently represents an integer from 3-6, such as 3, 4, 5 or 6, where the compound is crosslinked with a crosslinking agent as defined above in this paragraph. A further pharmaceutical composition embodiment of the present invention comprises an amine polymer comprising at least one amido-amine compound or residue thereof or at least one amido-amine dendrimer or residue thereof, where the amido-amine compound or amido-amine dendrimer is represented by Formula III where R₆ independently represents a H radical or alkyl radical, q, r and p are each 2, m independently represents an integer from 3-6, such as 3, 4, 5 or 6, where the compound is crosslinked with a crosslinking agent as defined above in this paragraph.

In some embodiments, the pharmaceutical composition of the present invention comprises an amido-amine polymer comprising at least one amido-amine compound or residue thereof or at least one amido-amine dendrimer or residue thereof, where the amido-amine compound or amido-amine dendrimer is represented by Formula VIII where R₆ independently represents a H radical or alkyl radical, q and r are 0 and p is 2, m independently represents an integer from 3-6, such as 3, 4, 5 or 6; and 2-6 wt. % crosslinking agent or residue thereof, such as 2 wt. %, 3 wt. %, 4 wt. %, 5 wt. % or 6 wt. % crosslinking agent, where the crosslinking agent is epichlorohydrin, poly(epichlorohydrin), 1,2-dibromoethane, tris(2-chloroethyl)amine or 1,4-butanediol diglycidyl ether. Another pharmaceutical composition embodiment of the present invention comprises an amido-amine polymer comprising at least one amido-amine compound or residue thereof or at least one amido-amine dendrimer or residue thereof, where the amido-amine compound or amido-amine dendrimer is represented by Formula VIII, where R₆ independently represents a H radical or alkyl radical, q is 0 and r and p both are 2, m independently represents an integer from 3-6, such as 3, 4, 5 or 6, where the compound is crosslinked with a crosslinking agent as defined above in this paragraph. A further pharmaceutical composition embodiment of the present invention comprises an amido-amine polymer comprising at least one amido-amine compound or residue thereof or at least one amido-amine dendrimer or residue thereof, where the amido-amine compound or amido-amine dendrimer is represented by Formula VIII where R₆ independently represents a H radical or alkyl radical, q, r and p are each 2, m independently represents an integer from 3-6, such as 3, 4, 5 or 6, where the compound is crosslinked with a crosslinking agent as defined above in this paragraph.

Another pharmaceutical composition of the present invention comprises an amido-amine polymer comprising at least one amido-amine compound or residue thereof or at least one amido-amine dendrimer or residue thereof, where the amido-amine compound or amido-amine dendrimer is derived from compounds represented by Formulas II and XI, where R₂ independently represents a H radical or a methyl radical and R represents a C₃-C₆ radical, where the amido-amine polymer is crosslinked with 2-6 wt. % crosslinking agent, such as 2 wt. %, 3 wt. %, 4 wt. %, 5 wt. % or 6 wt. % crosslinking agent, where the crosslinking agent is epichlorohydrin, poly(epichlorohydrin), 1,2-dibromoethane, tris(2-chloroethyl)amine or 1,4-butanediol diglycidyl ether. Another pharmaceutical composition embodiment of the present invention comprises an amido-amine polymer comprising at least one amido-amine compound or residue thereof or at least one amido-amine dendrimer or residue thereof, where the amido-amine compound or amido-amine dendrimer is derived from compounds represented by Formulas II and XII, where R₂ independently represents a H radical or a methyl radical and R represents a C₃-C₆ radical, where the amido-amine polymer is crosslinked with a crosslinking agent as defined above in this paragraph. A further pharmaceutical composition embodiment of the present invention comprises an amido-amine polymer comprising at least one amido-amine compound or residue thereof or at least one amido-amine dendrimer or residue thereof, where the amido-amine compound or amido-amine dendrimer is derived from compounds represented by Formulas II and XIII, where R₂ independently represents a H radical or a methyl radical and R represents a C₃-C₆ radical, where the amido-amine polymer is crosslinked with a crosslinking agent as defined above in this paragraph.

The polymers of some embodiments may be formed using a polymerization initiator. Generally, any initiator may be used including cationic and radical initiators. Some examples of suitable initiators that may be used include: the free radical peroxy and azo type compounds, such as azodiisobutyronitrile, azodiisovaleronitrile, dimethylazodiisobutyrate, 2,2′-azobis(isobutyronitrile), 2,2′-azobis(N,N′-dimethyleneisobutyramidine)dihydrochloride, 2,2′-azobis(2-amidinopropane)dihydrochloride, 2,2′-azobis(N,N′-dimethyleneisobutyramidine), 1,1′-azobis(1-cyclohexanecarbo-nitrile), 4,4′-azobis(4-cyanopentanoic acid), 2,2′-azobis(isobutyramide) dihydrate, 2,2′-azobis(2-methylpropane), 2,2′-azobis(2-methylbutyronitrile), VAZO 67, cyanopentanoic acid, the peroxy pivalates, dodecylbenzene peroxide, benzoyl peroxide, di-t-butyl hydroperoxide, t-butyl peracetate, acetyl peroxide, dicumyl peroxide, cumyl hydroperoxide, dimethyl bis(butylperoxy) hexane.

In some embodiments, any of the nitrogen atoms within the amido-amine compounds or residues thereof according to embodiments of the invention may optionally be quaternized to yield the corresponding positively charged tertiary nitrogen group, such as for example, an ammonium or substituted ammonium group. Any one or more of the nitrogen atoms in the amido-amine compound or residue thereof may be quaternized and such quaternization, when present, is not limited to or required to include terminal amine nitrogen atoms. In some embodiments, this quaternization may result in additional network formation and may be the result of addition of crosslinking, linking or amine reactive groups to the nitrogen. The ammonium groups may be associated with a pharmaceutically acceptable counterion.

In some embodiments, amido-amine compounds and amido-amine polymers of the invention may be partially or fully quaternized, including protonated, with a pharmaceutically acceptable counterion, which may be organic ions, inorganic ions, or a combination thereof. Examples of some suitable inorganic ions include halides (e.g., chloride, bromide or iodide) carbonates, bicarbonates, sulfates, bisulfates, hydroxides, nitrates, persulfates and sulfites. Examples of some suitable organic ions include acetates, ascorbates, benzoates, citrates, dihydrogen citrates, hydrogen citrates, oxalates, succinates, tartrates, taurocholates, glycocholates, and cholates. Preferred ions include chlorides and carbonates.

In some embodiments, amido-amine compounds and amido-amine polymers of the invention may be protonated such that the fraction of protonated nitrogen atoms is from 1 to 25%, preferably 3 to 25%, more preferably 5 to 15%.

In one embodiment, a pharmaceutically acceptable amido-amine polymer is an amido-amine polymer in protonated form and comprises a carbonate anion. In one embodiment the pharmaceutically acceptable amido-amine polymer is in protonated form and comprises a mixture of carbonate and bicarbonate anions.

In some embodiments, compounds of the invention are characterized by their ability to bind compounds or ions. Preferably the compounds of the invention bind anions, more preferably they bind organophosphates, phosphate and/or oxalate, and most preferably they bind organophosphates or phosphate. For illustration, anion-binding amido-amine polymers and especially organophosphate or phosphate-binding amido-amine polymers will be described; however, it is understood that this description applies equally, with appropriate modifications that will be apparent to those of skill in the art, to other ions, compounds and solutes. Amido-amine polymers may bind an ion, e.g., an anion when they associate with the ion, generally though not necessarily in a noncovalent manner, with sufficient association strength that at least a portion of the ion remains bound under the in vitro or in vivo conditions in which the polymer is used for sufficient time to effect a removal of the ion from solution or from the body. A target ion may be an ion to which the amido-amine polymer binds, and usually refers to the ion whose binding to the amido-amine polymer is thought to produce the therapeutic effect of the compound and may be an anion or a cation. A compound of the invention may have more than one target ion.

For example, some of the amido-amine polymers described herein exhibit organophosphate or phosphate binding properties. Phosphate binding capacity is a measure of the amount of phosphate ion a phosphate binder can bind in a given solution. For example, binding capacities of phosphate binders can be measured in vitro, e.g., in water or in saline solution, or in vivo, e.g., from phosphate urinary excretion, or ex vivo, for example using aspirate liquids, e.g., chyme obtained from lab animals, patients or volunteers. Measurements can be made in a solution containing only phosphate ion, or at least no other competing solutes that compete with phosphate ions for binding to the amido-amine polymer. In these cases, a non interfering buffer may be used. Alternatively, measurements can be made in the presence of other competing solutes, e.g., other ions or metabolites that compete with phosphate ions (the target solute) for binding to the amido-amine polymer.

Ion binding capacity for an amido-amine polymer may be measured as indicated in the Test Methods. Some embodiments have a phosphate binding capacity which can be greater than about 0.2, 0.5, 1.0, 1.5, 2.0, 2.5, 3.0, 3.5, 4.0, 5.0, 6.0, 8.0, 10.0, 12, 14, 16, 18 or greater than about 20 mmol/g. In some embodiments, the in vitro phosphate binding capacity of amido-amine polymers of the invention for a target ion is greater than about 0.5 mmol/g, preferably greater than about 2.5 mmol/g, even more preferably greater than about 3 mmol/g, even more preferably greater than about 4 mmol/g, and yet even more preferably greater than about 6 mmol/g. In some embodiments, the phosphate binding capacity can range from about 0.2 mmol/g to about 20 mmol/g, such as about 0.5 mmol/g to about 10 mmol/g, preferably from about 2.5 mmol/g to about 8 mmol/g, and even more preferably from about 3 mmol/g to about 6 mmol/g. Phosphate binding may be measured according to the techniques described in the Test Methods section below.

In some embodiments, amido-amine compounds, polymers and compositions of the invention may reduce urinary phosphorous of a patient in need thereof by 5-100%, such as 10-75%, 25-65%, or 45-60%. Some embodiments may reduce urinary phosphorous by greater than 10%, greater than 20%, greater than 30%, greater than 40%, greater than 45%, greater than 50% or greater than 60%. Reduction of urinary phosphorous may be measured according to the methods detailed in the Test Methods section below.

In some embodiments, amido-amine polymers and compositions of the invention may reduce blood phosphate of a patient in need thereof by 5-100%, such as 10-75%, 25-65%, or 45-60%. Some embodiments may reduce blood phosphate levels by greater than 10%, greater than 20%, greater than 30%, greater than 40%, greater than 45%, greater than 50% or greater than 60%.

When crosslinked, some embodiments of the amido-amine compounds of the invention form a gel in a solvent, such as in a simulated gastrointestinal medium or a physiologically acceptable medium.

One aspect of the invention is core-shell compositions comprising a polymeric core and shell. In some embodiments, the polymeric core comprises the amido-amine polymers described herein. The shell material can be chemically anchored to the core material or physically coated. In the former case, the shell can be grown on the core component through chemical means, for example by: chemical grafting of shell polymer to the core using living polymerization from active sites anchored onto the core polymer; interfacial reaction, i.e., a chemical reaction located at the core particle surface, such as interfacial polycondensation; and using block copolymers as suspending agents during the core particle synthesis.

In some embodiments, the interfacial reaction and use of block polymers are the techniques used when chemical methods are used. In the interfacial reaction pathway, typically, the periphery of the core particle is chemically modified by reacting small molecules or macromolecules on the core interface. For example, an amine containing ion-binding core particle is reacted with a polymer containing amine reactive groups such as epoxy, isocyanate, activated esters, halide groups to form a crosslinked shell around the core.

In another embodiment, the shell is first prepared using interfacial polycondensation or solvent coacervation to produce capsules. The interior of the capsule is then filled up with core-forming precursors to build the core within the shell capsule.

In some embodiments, using the block copolymer approach, an amphiphilic block copolymer can be used as a suspending agent to form the core particle in an inverse or direct suspension particle forming process. When an inverse water-in-oil suspension process is used, then the block copolymer comprises a first block soluble in the continuous oil phase and another hydrophilic block contains functional groups that can react with the core polymer. When added to the aqueous phase, along with core-forming precursor, and the oil phase, the block copolymer locates to the water-in-oil interface and acts as a suspending agent. The hydrophilic block reacts with the core material, or co-reacts with the core-forming precursors. After the particles are isolated from the oil phase, the block copolymers form a thin shell covalently attached to the core surface. The chemical nature and length of the blocks can be varied to vary the permeation characteristics of the shell towards solutes of interest.

When the shell material is physically adsorbed on the core material, well known techniques of microencapsulation such as solvent coacervation, fluidized bed spray coater, or multiemulsion processes can be used. One method of microencapsulation is the fluidized bed spray coater in the Wurster configuration. In yet another embodiment, the shell material is only acting temporarily by delaying the swelling of the core particle while in the mouth and esophagus, and optionally disintegrates in the stomach or duodenum. The shell is then selected in order to hinder the transport of water into the core particle, by creating a layer of high hydrophobicity and very low liquid water permeability.

In one embodiment the shell material carries negative charges while being in the milieu of use. Not being limited to one mechanism of action, it is thought that negatively charged shell material coated on anion-binding beads enhance the binding of small inorganic ions with a low charge density (such as phosphate) over competing ions with greater valency or size. Competing anions such as citrate, bile acids and fatty acids among others, may thus have a lesser relative affinity to the anion binding core possibly as a result of their limited permeability across the shell.

In some embodiments, shell materials are polymers carrying negative charges in the pH range typically found in the intestine. Examples include, but are not limited to, polymers that have pendant acid groups such as carboxylic, sulfonic, hydrosulfonic, sulfamic, phosphoric, hydrophosphoric, phosphonic, hydrophosphonic, phosphoramidic, phenolic, boronic and a combination thereof. The polymer can be protonated or unprotonated; in the latter case the acidic anion can be neutralized with pharmaceutically acceptable cations such as Na, K, Li, Ca, Mg, and NH₄.

In another embodiment the polyanion can be administered as a precursor that ultimately activates as a polyanion: for instance certain labile ester or anhydride forms of either polysulfonic or polycarboxylic acids are prone to hydrolysis in the acidic environment of the stomach and can convert to the active anions.

The shell polymers can be either linear, branched, hyperbranched, segmented (i.e. backbone polymer arranged in sequence of contiguous blocks of which at least one contains pendant acidic groups), comb-shaped, star-shaped or crosslinked in a network, fully and semi-interpenetrated network (LPN). The shell polymers are either random or blocky in composition and either covalently or physically attached to the core material. Examples of such shell polymers include, but are not limited to acrylic acid homopolymers or copolymers, methacrylic acid homopolymers or copolymers, and copolymers of methacrylate and methacrylic acid. Examples of such polymers are copolymers of methyl methacrylate and methacrylic acid and copolymers of ethyl acrylate and methacrylic acid, sold under the tradename Eudragit (Rohm GmbH & Co. KG): examples of which include Eudragit L100-55 and Eudragit L100 (a methyl methacrylate-methacrylic acid (1:1) copolymer, Degussa/Rohm), Eudragit L30-D55, Eudragit S 100-55 and Eudragit FS 30D, Eudragit S 100 (a methyl methacrylate-methacrylic acid (2:1) copolymer), Eudragit LD-55 (an ethyl acrylate-methacrylic acid (1:1) copolymer), copolymers of acrylates and methacrylates with quaternary ammonium groups, sold under the tradenames Eudragit RL and Eudragit RS, and a neutral ester dispersion without any functional groups, sold under the tradename Eudragit NE30-D.

Additional shell polymers include: poly(styrene sulfonate), Polycarbophil®; Polyacrylic acid(s); carboxymethyl cellulose, cellulose acetate phthalate, hydroxypropyl methylcellulose phthalate as sold under the tradename HP-50 and HP-55 (Shin-Etsu Chemical Co., Ltd.), cellulose acetate trimellitate, cellulose acetate, cellulose acetate butyrate, cellulose acetate propionate, ethyl cellulose, cellulose derivatives, such as hydroxypropylmethylcellulose, methylcelluose, hydroxylethylcellulose, hydroxyethylmethylcellulose, hydroxylethylethylcelluose and hydroxypropylethylcellulose and cellulose derivatives such as cellulose ethers useful in film coating formulations, polyvinyl acetate phthalate, carrageenan, alginate, or poly(methacrylic acid) esters, acrylic/maleic acid copolymers, styrene/maleic acid polymers, itaconic acid/acrylic copolymers, and fumaric/acrylic acid copolymers, polyvinyl acetal diethylaminoacetate, as sold under the tradename AEA (Sankyo Co., Ltd.), methylvinylether/maleic acid copolymers and shellac.

In some embodiments the shell polymers are selected amongst pharmaceutically acceptable polymers such as Eudragit L100-55 and Eudragit L100 (a methylmethacrylate-methacrylic acid (1:1) copolymer, Degussa/Rohm), Carbopol 934 (polyacrylic acid, Noveon), C-A-P NF (cellulose acetate phthalate—Eastman), Eastacryl (methacrylic acid esters—Eastman), Carrageenan and Alginate (FMC Biopolymer), Anycoat—P (Samsung Fine Chemicals—HPMC Phthalate), or Aqualon (carboxymethyl cellulose—Hercules), methylvinyletherimaleic acid copolymers (Gantrez), and styrene/maleic acid (SMA).

The shell can be coated by a variety of methods. In one embodiment, the shell materials are added in the drug formulation step as an active excipient; for example, the shell material can be included in a solid formulation as a powder, which is physically blended with the organophosphate or phosphate-binding polymer and other excipients, optionally granulated, and compressed to form a tablet. Thus, in some embodiments, the shell material need not cover the core material in the drug product. For example, the acidic shell polymer may be added together with the anion binding core polymer formulated in the shape of a tablet, capsule, gel, liquid, etc, wafer, extrudates and the shell polymer can then dissolve and distribute itself uniformly as a shell coating around the core while the drug product equilibrates in the mouth, esophagus or ultimately in the site of action, i.e. the GI tract.

In some embodiments, the shell is a thin layer of shell polymer. The layer can be a molecular layer of polyanion on the core particle surface. The weight to core ratio can be between about 0.0001% to about 30%, preferably comprised between about 0.01% to about 5%, such as between about 0.1% to about 5%.

The shell polymers have a minimum molecular weight such that they do not freely permeate within the core pore volume nor elute from the core surface. In some embodiments, the molecular weight (Mw) of the shell acidic polymer is above about 1000 g/mole, such as above about 5000 g/mole, and or even above about 20,000 g/mole

The anionic charge density of the shell material (as prevailing in the milieu of use) is may be between 0.5 mEq/gr to 22 mEq/gr, such as 2 mEq/gr to 15 mEq/gr. If a coating process is used to form the shell on the amido-amine polymer particles as part of the manufacture of the dosage form, then procedures known from those skilled-in-the-art in the pharmaceutical industry are applicable. In one embodiment, the shell is formed in a fluidized bed coater (Wurster coater). In an alternate embodiment, the shell is formed through controlled precipitation or coascervation, wherein the amido-amine polymer particles are suspended in a polymer solution, and the solvent properties are changed in such a way as to induce the polymer to precipitate onto or coat the amido-amine polymer particles.

Suitable coating processes include the procedures typically used in the pharmaceutical industry. Typically, selection of the coating method is dictated by a number of parameters, that include, but are not limited to the form of the shell material (bulk, solution, emulsion, suspension, melt) as well as the shape and nature of the core material (spherical beads, irregular shaped, etc.), and the amount of shell deposited. In addition, the cores may be coated with one or more shells and may comprise multiple or alternating layers of shells.

The term “phosphate imbalance disorder” as used herein refers to conditions in which the level of phosphorus present in the body is abnormal. One example of a phosphate imbalance disorder includes hyperphosphatemia. The term “hyperphosphatemia” as used herein refers to a condition in which the element phosphorus is present in the body at an elevated level. Typically, a patient is often diagnosed with hyperphosphatemia if the blood phosphate level is, for example, above about 4.0 or 4.5 milligrams per deciliter of blood, for example above about 5.0 mg/dl, such as above about 5.5 mg/dl, for example above 6.0 mg/dl, and/or a severely impaired glomerular filtration rate such as, for example, less than about 20% of normal. The present invention may also be used to treat patients suffering from hyperphosphatemia in End Stage Renal Disease and who are also receiving dialysis treatment (e.g., hemodialysis or peritoneal dialysis).

Other diseases that can be treated with the methods, compounds, polymers, compositions and kits of the present invention include hypocalcemia, hyvperparathyroidism, depressed renal synthesis of calcitriol, tetany due to hypocalcemia, renal insufficiency, and ectopic calcification in soft tissues including calcifications in joints, lungs, kidney, conjuctiva, and myocardial tissues. Also, the present invention can be used to treat Chronic Kidney Disease (CKD), End Stage Renal Disease (ESRD) and dialysis patients, including prophylactic treatment of any of the above.

The amido-amine polymers and compositions described herein can be used as an adjunct to other therapies e.g. those employing dietary control of phosphorus intake, dialysis, inorganic metal salts and/or other polymer resins.

The compositions of the present invention are also useful in removing chloride, bicarbonate, oxalate, and bile acids from the gastrointestinal tract. Amido-amine polymers removing oxalate compounds or ions find use in the treatment of oxalate imbalance disorders, such as oxalosis or hyperoxaluria that increases the risk of kidney stone formation. Amido-amine polymers removing chloride compounds or ions find use in treating acidosis, heartburn, acid reflux disease, sour stomach or gastritis, for example. In some embodiments, the compositions of the present invention are useful for removing fatty acids, bilirubin, and related compounds. Some embodiments may also bind and remove high molecular weight molecules like proteins, nucleic acids, vitamins or cell debris.

The present invention provides methods, pharmaceutical compositions, and kits for the treatment of animals. The term “animal” or “animal subject” or “patient” as used herein includes humans as well as other mammals (e.g., in veterinary treatments, such as in the treatment of dogs or cats, or livestock animals such as pigs, goats, cows, horses, chickens and the like). One embodiment of the invention is a method of removing phosphorous-containing compounds such as organophosphates or phosphate from the gastrointestinal tract, such as the stomach, small intestine or large intestine of an animal by administering an effective amount of at least one of the amido-amine polymers described herein.

The term “treating” and its grammatical equivalents as used herein include achieving a therapeutic benefit and/or a prophylactic benefit. By therapeutic benefit is meant eradication, amelioration, or prevention of the underlying disorder being treated. For example, in a hyperphosphatemia patient, therapeutic benefit includes eradication or amelioration of the underlying hyperphosphatemia. Also, a therapeutic benefit is achieved with the eradication, amelioration, or prevention of one or more of the physiological symptoms associated with the underlying disorder such that an improvement is observed in the patient, notwithstanding that the patient may still be afflicted with the underlying disorder. For example, administration of amido-amine polymers, described herein, to a patient suffering from renal insufficiency and/or hyperphosphatemia provides therapeutic benefit not only when the patient's serum phosphate level is decreased, but also when an improvement is observed in the patient with respect to other disorders that accompany renal failure and/or hyperphosphatemia like ectopic calcification and renal osteodistrophy. For prophylactic benefit, for example, the amido-amine polymers may be administered to a patient at risk of developing hyperphosphatemia or to a patient reporting one or more of the physiological symptoms of hyperphosphatemia, even though a diagnosis of hyperphosphatemia may not have been made.

The compositions may also be used to control serum phosphate in subjects with elevated phosphate levels, for example, by changing the serum level of phosphate towards a normal or near normal level, for example, towards a level that is within 10% of the normal level of a healthy patient.

Other embodiments of the invention are directed towards pharmaceutical compositions comprising at least one of the amido-amine polymers or a pharmaceutically acceptable salt of the amido-amine polymer, and one or more pharmaceutically acceptable excipients, diluents, or carriers and optionally additional therapeutic agents. The compounds may be lyophilized or dried under vacuum or oven before formulating.

The excipients or carriers are “acceptable” in the sense of being compatible with the other ingredients of the formulation and not deleterious to the recipient thereof. The formulations can conveniently be presented in unit dosage form and can be prepared by any suitable method. The methods typically include the step of bringing into association the agent with the excipients or carriers such as by uniformly and intimately bringing into association the amido-amine polymer with the excipients or carriers and then, if necessary, dividing the product into unit dosages thereof.

The pharmaceutical compositions of the present invention include compositions wherein the amido-amine polymers are present in an effective amount, i.e., in an amount effective to achieve therapeutic and/or prophylactic benefit. The actual amount effective for a particular application will depend on the patient (e.g. age, weight, etc.) the condition being treated; and the route of administration.

The dosages of the amido-amine polymers in animals will depend on the disease being, treated, the route of administration, and the physical characteristics of the animal being treated. Such dosage levels in some embodiments for either therapeutic and/or prophylactic uses may be from about 1 gm/day to about 30 gm/day, for example from about 2 gm/day to about 20 gm/day or from about 3 gm/day to about 7 gm/day. The dose of the amido-amine polymers described herein can be less than about 50 gm/day, less than about 40 gm/day, less than about 30 gm/day, less than about 20 gm/day, and less than about 10 gm/day.

Typically, the amido-amine polymers can be administered before or after a meal, or with a meal. As used herein, “before” or “after” a meal is typically within two hours, preferably within one hour, more preferably within thirty minutes, most preferably within ten minutes of commencing or finishing a meal, respectively.

Generally, it is preferred that the amido-amine polymers are administered along with meals. The amido-amine polymers may be administered one time a day, two times a day, or three times a day. Preferably the amido-amine polymers are administered once a day with the largest meal.

Preferably, the amido-amine polymers may be used for therapeutic and/or prophylactic benefits and can be administered alone or in the form of a pharmaceutical composition. The pharmaceutical compositions comprise the amido-amine polymers, one or more pharmaceutically acceptable carriers, diluents or excipients, and optionally additional therapeutic agents. For example, the amido-amine polymers of the present invention may be co-administered with other active pharmaceutical agents depending on the condition being treated. Examples of pharmaceutical agents that may be co-administered include, but are not limited to:

Other phosphate sequestrants including pharmaceutically acceptable lanthanum, calcium, aluminum, magnesium and zinc compounds, such as acetates, carbonates, oxides, hydroxides, citrates, alginates, and ketoacids thereof.

Calcium compounds, including calcium carbonate, acetate (such as PhosLo®calcium acetate tablets), citrate, alginate, and ketoacids, have been utilized for phosphate binding.

Aluminium-based phosphate sequestrants, such as Amphojel® aluminium hydroxide gel, have also been used for treating hyperphosphatemia. These compounds complex with intestinal phosphate to form highly insoluble aluminium phosphate; the bound phosphate is unavailable for absorption by the patient.

The most commonly used lanthanide compound, lanthanum carbonate (Fosrenol®) behaves similarly to calcium carbonate.

Other phosphate sequestrants suitable for use in the present invention include pharmaceutically acceptable magnesium compounds. Various examples of pharmaceutically acceptable magnesium compounds are described in U.S. Provisional Application No. 60/734,593 filed Nov. 8, 2005, the entire teachings of which are incorporated herein by reference. Specific suitable examples include magnesium oxide, magnesium hydroxide, magnesium halides (e.g., magnesium fluoride, magnesium chloride, magnesium bromide and magnesium iodide), magnesium alkoxides (e.g., magnesium ethoxide and magnesium isopropoxide), magnesium carbonate, magnesium bicarbonate, magnesium formate, magnesium acetate, magnesium trisilicates, magnesium salts of organic acids, such as fumaric acid, maleic acid, acrylic acid, methacrylic acid, itaconic acid and styrenesulfonic acid, and a combination thereof.

Various examples of pharmaceutically acceptable zinc compounds are described in PCT Application No. PCT/US2005/047582 filed Dec. 29, 2005, the entire teachings of which are incorporated herein by reference. Specific suitable examples of pharmaceutically acceptable zinc compounds include zinc acetate, zinc bromide, zinc caprylate, zinc carbonate, zinc chloride, zinc citrate, zinc formate, zinc hexafluorosilicate, zinc iodate, zinc iodide, zinc iodide-starch, zinc lactate, zinc nitrate, zinc oleate, zinc oxalate, zinc oxide, calamine (zinc oxide with a small proportion of ferric oxide), zinc p-phenolsulfonate, zinc propionate, zinc salicylate, zinc silicate, zinc stearate, zinc sulfate, zinc sulfide, zinc tannate, zinc tartrate, zinc valerate and zinc ethylenebis(dithiocarbamate). Another example includes poly(zinc acrylate).

When referring to any of the above-mentioned phosphate sequestrants, it is to be understood that mixtures, polymorphs and solvates thereof are encompassed.

In some embodiments, a mixture of the phosphate sequestrants described above can be used in the invention in combination with pharmaceutically acceptable ferrous iron salts.

In other embodiments, the phosphate sequestrant used in combination with compounds of the present invention is not a pharmaceutically acceptable magnesium compound. In yet other embodiments, the phosphate sequestrant used in combination with the pharmaceutically acceptable amido-amine compounds and/or amido-amine polymers is not a pharmaceutically acceptable zinc compound.

The invention also includes methods and pharmaceutical compositions directed to a combination therapy of the amido-amine polymers in combination with a phosphate transport inhibitor or an alkaline phosphatase inhibitor. Alternatively, a mixture of the amido-amine polymers is employed together with a phosphate transport inhibitor or an alkaline phosphatase inhibitor.

Suitable examples of phosphate transport inhibitors can be found in co-pending U.S. Application Publication Nos. 2004/0019113 and 2004/0019020 and WO 2004/085448, the entire teachings of each of which are incorporated herein by reference.

A large variety of organic and inorganic molecules are inhibitors to alkaline phosphatase (ALP) (see, for example, U.S. Pat. No. 5,948,630, the entire teachings of which are incorporated herein by reference). Examples of alkaline phosphatase inhibitors include orthophosphate, arsenate, L-phenylalanine, L-homoarginine, tetramisole, levamisole, L-p-Bromotetramisole, 5,6-Dihydro-6-(2-naphthyl) imidazo-[2,1-b]thiazole (napthyl) and derivatives thereof. The preferred inhibitors include, but are not limited to, levamisole, bromotetramisole, and 5,6-Dihydro-6-(2-naphthyl)imidazo-[2,1-b]thiazole and derivatives thereof.

This co-administration can include simultaneous administration of the two agents in the same dosage form, simultaneous administration in separate dosage forms, and separate administration. For example, for the treatment of hyperphosphatemia, the amido-amine polymers may be co-administered with calcium salts which are used to treat hypocalcemia resulting from hyperphosphatemia.

The pharmaceutical compositions of the invention can be formulated as a tablet, sachet, slurry, food formulation, troche, capsule, elixir, suspension, syrup, wafer, chewing gum or lozenge.

Preferably, the amido-amine polymers or the pharmaceutical compositions comprising the amido-amine polymers are administered orally. Illustrative of suitable methods, vehicles, excipients and carriers are those described, for example, in Remington's Pharmaceutical Sciences, 19th ed., the contents of which is incorporated herein by reference.

Pharmaceutical compositions for use in accordance with the present invention may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Suitable techniques for preparing pharmaceutical compositions of the amido-amines are well known in the art.

In some aspects of the invention, the amido-amine polymer(s) provide mechanical and thermal properties that are usually performed by excipients, thus decreasing the amount of such excipients required for the formulation. In some embodiments the amido-amine polymer or composition constitutes over about 30 wt. %, for example over about 40 wt. %, over about 50 wt. %, preferably over about 60 wt. %, over about 70 wt. %, more preferably over about 80 wt. %, over about 85 wt. % or over about 90 wt. % of the composition, the remainder comprising suitable excipient(s).

In some embodiments, the compressibility of the tablets is strongly dependent upon the degree of hydration (moisture content) of the amido-amine polymer. Preferably, the amido-amine polymer has a moisture content of about 5% by weight or greater, more preferably, the moisture content is from about 5% to about 9% by weight, and most preferably about 7% by weight. It is to be understood that in embodiments in which the amido-amine polymer is hydrated, the water of hydration is considered to be a component of the amido-amine polymer.

The tablet can further comprise one or more excipients, such as hardeners, glidants and lubricants, which are well known in the art. Suitable excipients include colloidal silicon dioxide, stearic acid, magnesium silicate, calcium silicate, sucrose, calcium stearate, glyceryl behenate, magnesium stearate, talc, zinc stearate and sodium stearylfumarate.

The tablet core of embodiments of the invention may be prepared by a method comprising the steps of: (1) hydrating or drying the amido-amine polymer to the desired moisture level; (2) blending the amido-amine polymer with any excipients; and (3) compressing the blend using conventional tableting technology.

In some embodiments, the invention relates to a stable, swallowable coated tablet, particularly a tablet comprising a hydrophilic core, such as a tablet comprising the amido-amine polymer, as described above. In one embodiment, the coating composition comprises a cellulose derivative and a plasticizing agent. The cellulose derivative is, preferably, hydroxypropylmethylcellulose (HPMC). The cellulose derivative can be present as an aqueous solution. Suitable hydroxypropylmethylcellulose solutions include those containing HPMC low viscosity and/or HPMC high viscosity. Additional suitable cellulose derivatives include cellulose ethers useful in film coating formulations. The plasticizing agent can be, for example, an acetylated monoglyceride such as diacetylated monoglyceride. The coating composition can further include a pigment selected to provide a tablet coating of the desired color. For example, to produce a white coating, a white pigment can be selected, such as titanium dioxide.

In one embodiment, the coated tablet of the invention can be prepared by a method comprising the step of contacting a tablet core of the invention, as described above, with a coating solution comprising a solvent, at least one coating agent dissolved or suspended in the solvent and, optionally, one or more plasticizing agents. Preferably, the solvent is an aqueous solvent, such as water or an aqueous buffer, or a mixed aqueous/organic solvent. Preferred coating agents include cellulose derivatives, such as hydroxypropylmethylcellulose. Typically, the tablet core is contacted with the coating solution until the weight of the tablet core has increased by an amount ranging from about 4% to about 6%, indicating the deposition of a suitable coating on the tablet core to form a coated tablet.

Other pharmaceutical excipients useful in the some compositions of the invention include a binder, such as microcrystalline cellulose, carbopol, providone and xanthan gum; a flavoring agent, such as mannitol, xylitol, maltodextrin, fructose, or sorbitol; a lubricant, such as vegetable based fatty acids; and, optionally, a disintegrant, such as croscarmellose sodium, gellan gum, low-substituted hydroxypropyl ether of cellulose, sodium starch glycolate. Such additives and other suitable ingredients are well-known in the art; see, e.g., Gennaro A R (ed), Remington's Pharmaceutical Sciences, 19th Edition.

In some embodiments the amido-amine polymers of the invention are provided as pharmaceutical compositions in the form of chewable tablets. In addition to the active ingredient, the following types of excipients are commonly used: a sweetening agent to provide the necessary palatability, plus a binder where the former is inadequate in providing sufficient tablet hardness; a lubricant to minimize frictional effects at the die wall and facilitate tablet ejection; and, in some formulations a small amount of a disintegrant is added to facilitate mastication. In general excipient levels in currently-available chewable tablets are on the order of 3-5 fold of active ingredient(s) whereas sweetening agents make up the bulk of the inactive ingredients. In some embodiments the invention provides a pharmaceutical composition formulated as a chewable tablet, comprising an amido-amine polymer described herein, a filler, and a lubricant. In some embodiments the invention provides a pharmaceutical composition formulated as a chewable tablet, comprising an amido-amine polymer described herein, a filler, and a lubricant, wherein the filler is chosen from the group consisting of sucrose, mannitol, xylitol, maltodextrin, fructose, and sorbitol, and wherein the lubricant is a magnesium fatty acid salt, such as magnesium stearate.

In one embodiment, the amido-amine polymer is pre-formulated with a high Tg/high melting point low molecular weight excipient such as mannitol, sorbose, sucrose in order to form a solid solution wherein the polymer and the excipient are intimately mixed. Methods of mixing such as extrusion, spray-drying, chill drying, lyophilization, or wet granulation are useful. Indication of the level of mixing is given by known physical methods such as differential scanning calorimetry or dynamic mechanical analysis.

In some embodiments the amido-amine polymers of the invention are provided as pharmaceutical compositions in the form of liquid formulations. In some embodiments the pharmaceutical composition contains polymer dispersed in a suitable liquid excipient. Suitable liquid excipients are known in the art; see, e.g., Remington's Pharmaceutical Sciences.

In some embodiments, the pharmaceutical compositions may be in the form of a powder formulation packaged as a sachet that may be mixed with water or other ingestible liquid and administered orally as a drink (solution or suspension). In order to ensure that such formulations provide acceptable properties to the patient such as mouth feel and taste, a pharmaceutically acceptable anionic stabilizer may be included in the formulation.

Examples of suitable anionic stabilizers include anionic polymers such as: an anionic polypeptide, an anionic polysaccharide, or a polymer of one or more anionic monomers such as polymers of mannuronic acid, guluronic acid, acrylic acid, methacrylic acid, glucuronic acid glutamic acid or a combination thereof, and pharmaceutically acceptable salts thereof. Other examples of anionic polymers include cellulose, such as carboxyalkyl cellulose or a pharmaceutically acceptable salt thereof. The anionic polymer may be a homopoloymer or copolymer of two or more of the anionic monomers described above. Alternatively, the anionic copolymer may include one or more anionic monomers and one or more neutral comonomers such as olefinic anionic monomers such as vinyl alcohol, acrylamide, and vinyl formamide.

Examples of anionic polymers include alginates (e.g. sodium alginate, potassium alginate, calcium alginate, magnesium alginate, ammonium alginate, and esters of alginate), carboxymethyl cellulose, polylactic acid, polyglutamic acid, pectin, xanthan, carrageenan, furcellaran, gum Arabic, karaya gum, gum ghatti, gum carob, and gum tragacanth. Preferred anionic polymers are alginates and are preferably esterified alginates such as a C2-C5-diol ester of alginate or a C3-C5 triol ester of alginate. As used herein an “esterified alginate” means an alginic acid in which one or more of the carboxyl groups of the alginic acid are esterified. The remainder of the carboxylic acid groups in the alginate are optionally neutralized (partially or completely) as pharmaceutically acceptable salts. For example, propylene glycol alginate is an ester of alginic acid in which some of the carboxyl groups are esterified with propylene glycol, and the remainder of the carboxylic acid groups is optionally neutralized with pharmaceutically acceptable salts. More preferably, the anionic polymer is ethylene glycol alginate, propylene glycol alginate or glycerol alginate, with propylene glycol alginate even more preferred.

All publications and patent applications mentioned in this specification are herein incorporated by reference to the same extent as if each individual publication or patent application was specifically and individually indicated to be incorporated by reference.

It will be apparent to one of ordinary skill in the art that many changes and modification can be made to the disclosures presented herein without departing from the spirit or scope of the appended claims.

### EXAMPLES

As used herein, the following terms have the meanings ascribed to them unless specified otherwise:

PAMAM—A second generation starburst dendrimer having a diaminobutane core and 16 terminal amino groups was obtained from Dendritic Nanotechnologies, Inc.

### Materials Used

Methanol and epichlorohydrin are commercially available from Sigma-Aldrich, Co. and were used without further purification.

### Example 1

### Synthesis of Compound I

550 mg of PAMAM was added to 1.1 ml of deionized water and stirred. 20.96 μl of epichlorohydrin was added. A gel formed after stirring overnight at room temperature. The gel was broken into small pieces and suspended in 1.5 L of deionized water, filtered and dried in a forced air oven at 60° C.

### Example II

### Synthesis of Compound II

6 g of a 20% solution of PAMAM in methanol was concentrated on a rotary evaporator. 7 g of deionized water was added to the concentrated PAMAM solution and stirred. 153 μl of epichlorohydrin was added. A gel formed after stirring overnight at room temperature. The gel was broken into small pieces, suspended in 2 L of deionized water, stirred and filtered. The filtered material was resuspended in 2 L of deionized water, stirred and filtered. The filtered polymer having a wet weight of 55.9 g was dried in a forced air oven at 60° C. to yield 700 mg of the desired product having an in-process-swelling ratio of 78.86 ml/g.

### Test Methods

### Amido-Amine Polymer Urinary Phosphorous Reduction (In Vivo-Rats)

House male Sprague Dawley (SD) rats may be used for the experiments. The rats are placed singly in wire-bottom cages, fed with Purina 5002 diet, and allowed to acclimate for at least 5 days prior to experimental use.

To establish baseline phosphorus excretion, the rats are placed in metabolic cages for 48 hours. Their urine is collected and its phosphorus content analyzed with a Hitachi analyzer to determine phosphorus excretion in mg/day. Any rats with outlying values should be excluded; and the remainder of the rats is distributed into groups.

Purina 5002 may be used as the standard diet. The amido-amine polymer being tested is mixed with Purina 5002 to result in a final amido-amine polymer concentration of 0.25% by weight of the feed. Cellulose at 0.5% by weight is used as a negative control. Sevelamer at 0.5% by weight is used as a positive control. For each rat, 200 g of diet is prepared.

Each rat is weighed and placed on the standard diet. After 4 days the standard diet is replaced with the treatment diet (or control diet for the control group). On days 5 and 6, urine samples from the rats at 24 hours (+/−30 minutes) are collected and analyzed. The test rats are again weighed, and any weight loss or gain is calculated. Any remaining food is also weighed to calculate the amount of food consumed per day. A change in phosphorus excretion relative to baseline and cellulose negative control is calculated. Percentage reduction of urinary phosphorous may be determined by the following equation:

% Reduction of Urinary Phosphorous=[(urinary phosphorous of negative control(mg/day)−urinary phosphorous of experimental(mg/day))/urinary phosphorous of negative control(mg/day)]×100.

In Vitro Phosphate Binding (mmol/g)

Two samples per polymer are weighed into plastic bottles after having adjusted the weight of the polymer for the loss on drying of each sample. A 10 mM phosphate buffer solution containing 10 mM KH₂PO₄, 100 mM N,N-bis[2-hydroxyethyl]-2-aminoethanesulfonic acid, 80 mM NaCl. 15 mM glycochenodeoxycholic acid (GCDC), and 15 mM oleic acid (pH adjusted to 7.0 with 1 N NaOH) is prepared and well mixed. Aliquots of the 10 mM phosphate buffer solution are transferred into each of the two sample bottles. The solutions are well mixed and then placed into an orbital shaker at 37° C. for 1 hour. The polymer is allowed to settle prior to removing a sample aliquot from each solution. The sample aliquot is filtered into a small vial using a disposable syringe and syringe filter. The filtered sample is diluted 1-to-10 with DI water. The shaking is continued for a further 4 hours (total of 5 hours) and the sampling procedure is repeated. Phosphate standards are prepared from a 10 mM phosphate standard stock solution and diluted appropriately to provide standards in the range of 0.3 to 1.0 mM. Both the standards and samples are analyzed by ion chromatography. A standard curve is set up and the unbound phosphate (mM) for each test solution is calculated. Bound phosphate is determined by the following equation:

Bound Phosphate(mmol/g)=[(10−Unbound PO₄)×Vol.×1000]/MassP;wherein Vol.=volume of test solution(L);MassP=LOD adjusted mass of polymer(mg).

In-Process Swelling Ratio (ml/g)

The in-process swelling ratio (SR) of several examples may be determined by the following equation:

SR=(weight of wet gel(g)−weight of dry polymer(g))/weight of dry polymer(g).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-72. (canceled)

73. A pharmaceutical composition comprising:
a) an amido-amine polymer comprising an amido-amine dendrimer derived from:
(i) a multi-amine; and
(ii) a multifunctional compound comprising two or more amine-reactive groups; and
b) a pharmaceutically acceptable excipient.

74. The composition according to claim 73, wherein the amine reactive groups are independently selected from the group consisting of vinyl groups, carboxylic acid groups and ester groups and combinations thereof.

75. The composition according to claim 73, wherein the multifunctional compound is selected from the group consisting of: 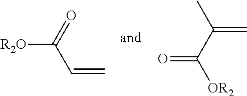
wherein R₂ independently represents a hydrogen radical or a branched or unbranched, substituted or un-substituted alkyl radical.

76. The composition of claim 73, wherein the dendrimer is formed using a series of alternating reactions.

77. The composition according to claim 73, wherein the multi-amine is selected from the group consisting of: 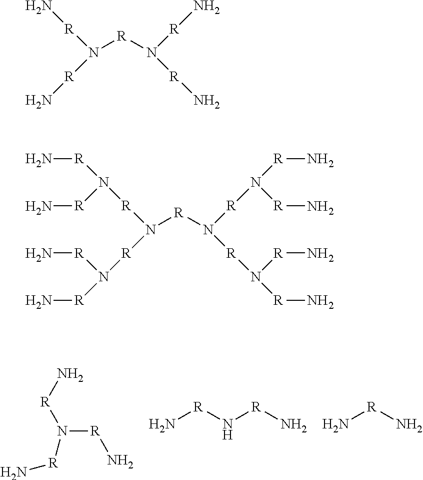
and combinations thereof, wherein R independently represents a branched or unbranched, substituted or un-substituted alkyl radical.

78. The composition according to claim 77, wherein the multi-amine is selected from the group consisting of: 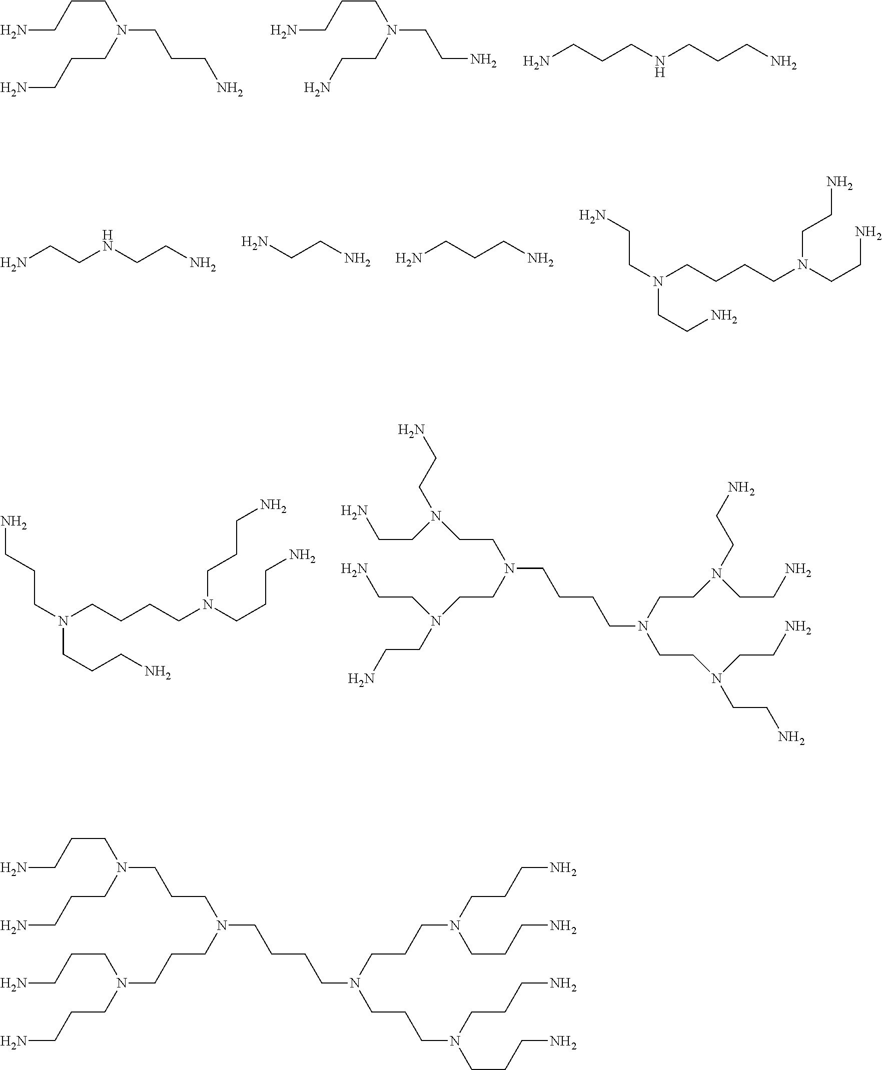
and combinations thereof.

79. The composition according to claim 73, wherein the multi-amine comprises a combination of more than one multi-amines selected from the group consisting of: 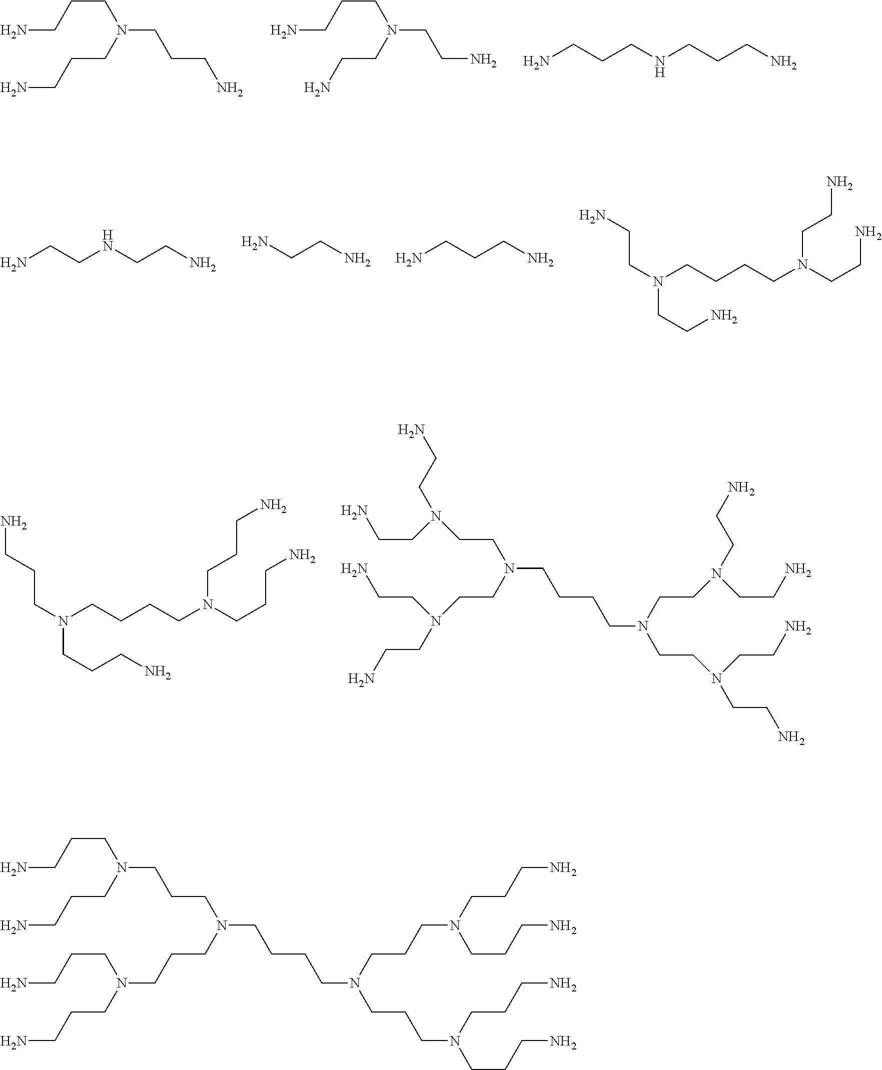
and combinations thereof.

80. The composition of claim 73, wherein the multi-amine comprises a combination of two multi-amines selected from the following groups:

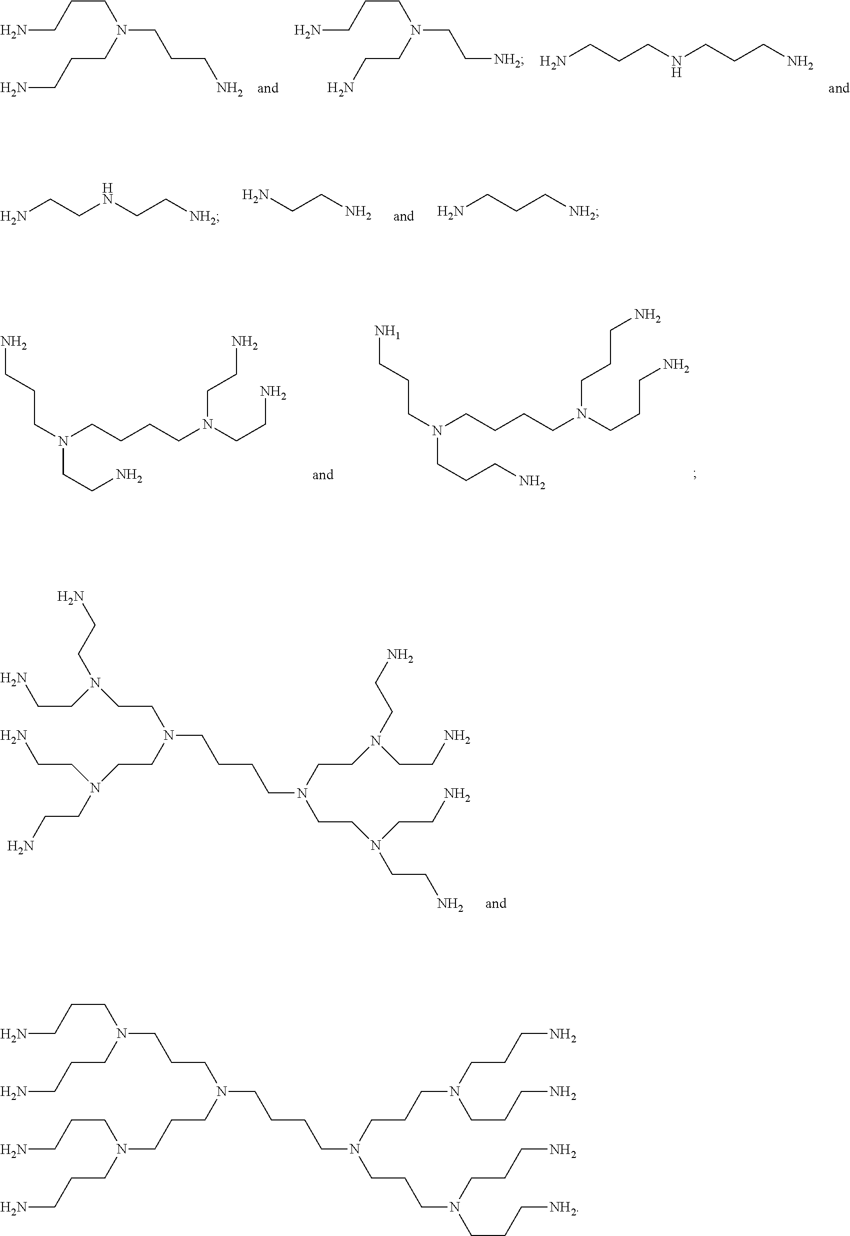

81. A pharmaceutical composition comprising:
a) at least one amido-amine polymer comprising at least one amido-amine dendrimer or residue thereof, said amido-amine dendrimer represented by the following Formula III: 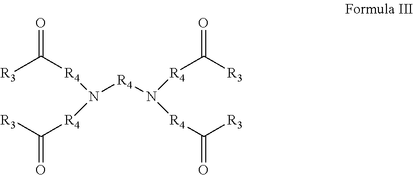
wherein R3 independently represents a group represented by the following Formula IV 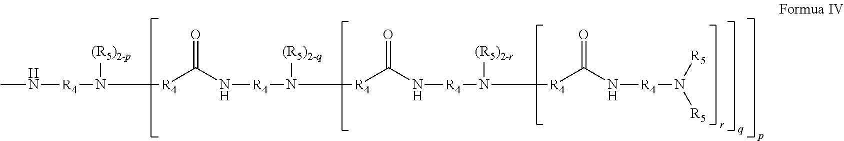
wherein
p, q and r independently represent an integer from 0-2;
R₄ independently represents 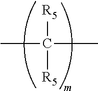
wherein
m independently represents an integer from 1-20;
R₅ independently represents a hydrogen radical; a substituted or unsubstituted alkyl radical; a substituted or un-substituted aryl radical; or
R₅ and a neighboring R₅ together represent a link or links comprising a residue of a crosslinking agent, a substituted or un-substituted alicyclic radical, a substituted or un-substituted aromatic radical, or a substituted or un-substituted heterocyclic radical; or R₅ represents a link with another compound or a residue thereof;
b) a crosslinking agent or residue thereof; and
c) a pharmaceutically acceptable excipient.

82. The composition of claim 81, wherein the amido-amine dendrimer is represented by the following Formula VI: 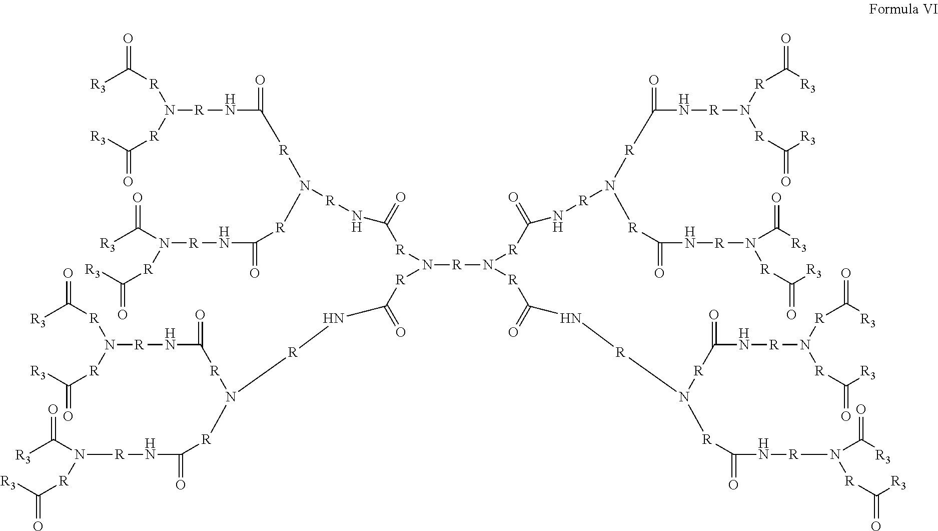
wherein R independently represents a branched or unbranched, substituted or un-substituted alkyl radical.

83. A pharmaceutical composition comprising:
a) at least one polymer comprising at least one amido-amine dendrimer or residue thereof, said amido-amine dendrimer represented by the following Formula VIII: 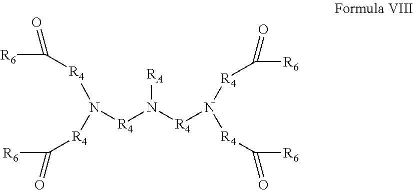
wherein
R₅ independently represents a group represented by the following Formula IX: wherein 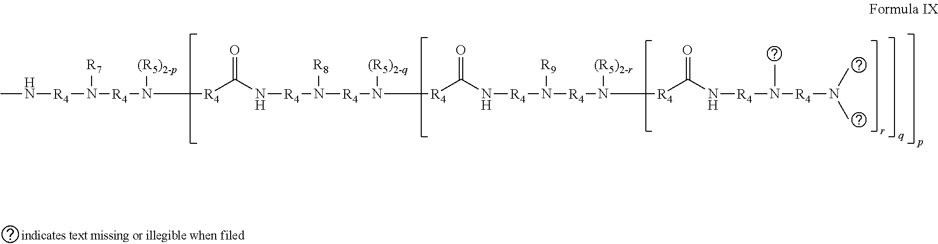
wherein
p, q and r independently represent an integer from 0-2;
R₄ independently represents: 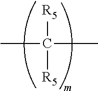
where m independently represents an integer from 1-20;
R₅ independently represents a hydrogen radical; a substituted or unsubstituted alkyl radical; a substituted or un-substituted aryl radical; or
R₅ and a neighboring R₅ together represent a link or links comprising a residue of a crosslinking agent, a substituted or un-substituted alicyclic radical, a substituted or un-substituted aromatic radical, or a substituted or unsubstituted heterocyclic radical; or R₅ represents a link with another compound or a residue thereof;
R_{A} independently represents an R₅ group or a —R₄—CO—R₆ group;
R₇ independently represents an R₅ group or independently represents a group according to the following Formula XIV: 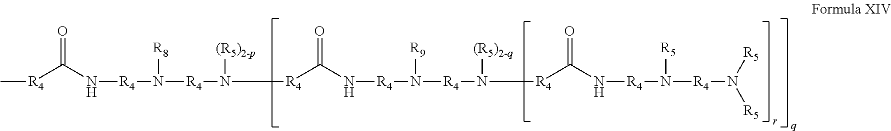
R₈ independently represents an Rs group or independently represents a group according to the following Formula XV: 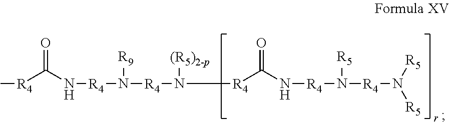
R₉ independently represents an R₅ group or independently represents a group according to the following Formula XVI: 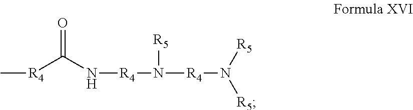
b) a crosslinking agent or residue thereof; and
c) a pharmaceutically acceptable excipient.

84. The composition of claim 83, wherein the amido-amine dendrimer is represented by the following Formula X: 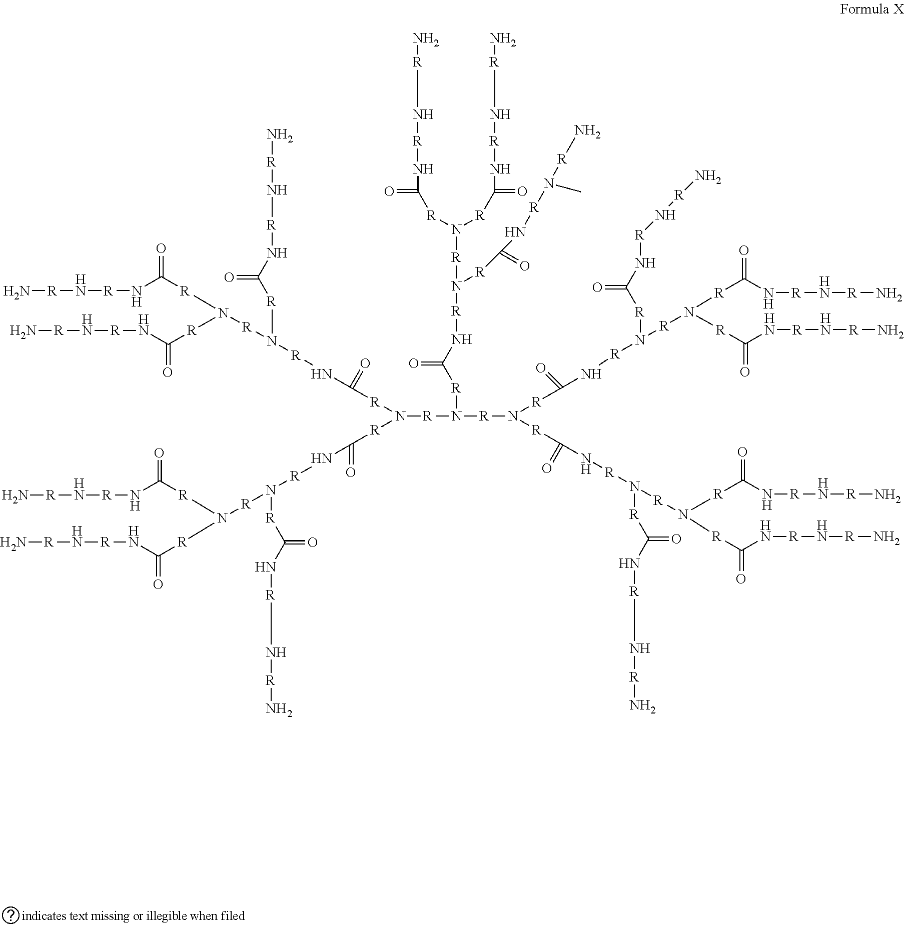
wherein R independently represents a branched or unbranched, substituted or unsubstituted alkyl radical.

85. A method of treating hyperphosphatemia, hypocalcemia, hyperparathyroidism, depressed renal synthesis of calcitriol, tetany due to hypocalcemia, renal insufficiency, and ectopic calcification in soft tissues including calcifications in joints, lungs, kidney, conjuctiva, and myocardial tissues, chronic kidney disease, ESRD and dialysis patients comprising administering to a patient in need thereof a therapeutically effective amount of at least one polymer comprising at least one amido-amine dendrimer or residue thereof, said amido-amine dendrimer derived from compounds represented by the following Formulas II and XI: 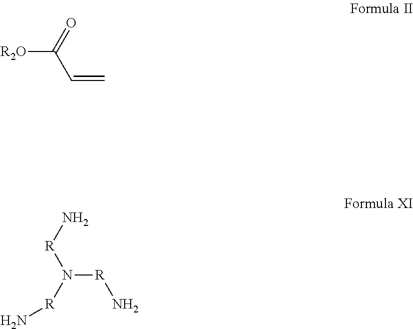
wherein R independently represents a branched or unbranched, substituted or un-substituted alkyl radical; R₂ independently represents a hydrogen radical or a branched or unbranched, substituted or un-substituted alkyl radical; and
a pharmaceutically acceptable excipient.

86. A method of treating hyperphosphatemia, hypocalcemia, hyperparathyroidism, depressed renal synthesis of calcitriol, tetany due to hypocalcemia, renal insufficiency, and ectopic calcification in soft tissues including calcifications in joints, lungs, kidney, conjuctiva, and myocardial tissues, chronic kidney disease, ESRD and dialysis patients comprising administering to a patient in need thereof a therapeutically effective amount of at least one polymer comprising at least one amido-amine dendrimer or residue thereof, said amido-amine dendrimer derived from compounds represented by the following Formulas II and XII: 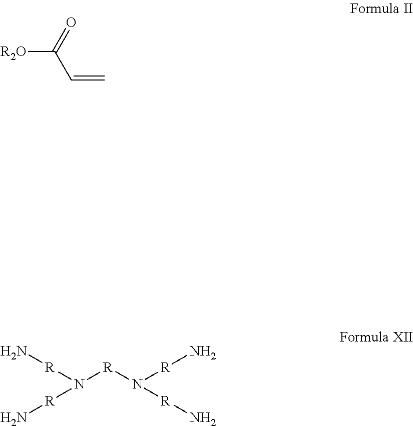
wherein R independently represents a branched or unbranched, substituted or unsubstituted alkyl radical; R₂ independently represents a hydrogen radical or a branched or unbranched, substituted or un-substituted alkyl radical; and
